(19) United States
(12) Patent Application Publication
(10) Pub. No.: US 2026/0061106 A1
(43) Pub. Date: Mar. 5, 2026

(54) METHODS OF REMOVING PARTICLES BY PLASMAPHERESIS

(71) Applicant: Circulate Health, Inc., Seattle, WA (US)

(72) Inventors: Brad Younggren, Seattle, WA (US); Dobri Kiprov, Menlo Park, CA (US)

(21) Appl. No.: 19/381,974

(22) Filed: Nov. 6, 2025

Related U.S. Application Data
(63) Continuation of application No. PCT/US2025/045238, filed on Sep. 5, 2025.
(60) Provisional application No. 63/691,276, filed on Sep. 5, 2024.

(51) Int. Cl. A61M 1/34

(52) U.S. Cl. CPC A61M 1/3496; A61M 1/342; A61M 2202/06; A61M 2205/3327; A61M 2205/7545

(57) ABSTRACT

The present disclosure provides methods and systems for removing particles from an individual using plasmapheresis, including from an individual's circulatory system, blood vessels, and/or organs. In various embodiments, the particles removed are plastic particles. In further embodiments, the present disclosure provides methods for treatment by reducing levels of particles in an individual's circulatory system, blood vessels, and/or organs.

### CROSS-REFERENCE TO RELATED APPLICATIONS

This application is a continuation of International Application No. PCT/US2025/045238, filed Sep. 5, 2025, which claims the benefit of priority under 35 U.S.C. § 119 (e) of U.S. Provisional Application No. 63/691,276 filed Sep. 5, 2024. The disclosure of the prior applications is considered part of and is herein incorporated by reference in the disclosure of this application in its entirety.

### BACKGROUND OF THE DISCLOSURE

Nanoparticle pollution has become a global concern in recent decades, with the production and disposal of synthetic materials increasing dramatically. Plastic particles have been found in every environment, and we are only now beginning to understand some of the health risks of these contaminants. As larger plastic debris degrades in the environment, it breaks down into smaller particles known as microplastic particles (less than 5 mm in size) and plastic particles (ranging from 1 nm to 1000 nm). These tiny plastic particles have been detected in various ecosystems, including marine environments, freshwater systems, and terrestrial habitats. Other micro- and nanoparticles are detrimental as well, such as particles from car tires and wildfires. The potential health impacts of small particle exposure for humans and other organisms are not yet fully understood. New methods are needed to address the health impacts of micro-and nano-particle exposure in humans and other organisms.

### SUMMARY OF THE DISCLOSURE

In various aspects, the present disclosure provides a method of reducing a level of exogenous particles in an individual by plasmapheresis, the method comprising: (a) withdrawing a volume of whole blood from the individual having a pre-treatment level of exogenous particles; (b) separating the volume of whole blood into a cellular fraction and an exogenous particle fraction, wherein the exogenous particle fraction comprises a level of exogenous particles; and (c) returning the cellular fraction and an exchange fluid to a circulatory system of the individual, thereby reducing the pre-treatment level of exogenous particles to a post-treatment level of exogenous particles in the individual.

In various aspects, the present disclosure provides a method of treating a condition associated with exposure to exogenous particles in an individual in need thereof, the method comprising: (a) measuring a pre-treatment level of the exogenous particles in a blood sample of the individual prior to performing plasmapheresis; (b) withdrawing a volume of whole blood from the individual; (c) separating the volume of whole blood into a cellular fraction and an exogenous particle fraction, wherein the exogenous particle fraction comprises a level of exogenous particles; and (d) returning the cellular fraction and an exchange fluid to a circulatory system of the individual, thereby reducing the pre-treatment level of exogenous particles to a post-treatment level of exogenous particles in the individual.

In various aspects, the present disclosure provides a method of providing plasmapheresis to an individual for reducing a level of exogenous particles in the individual, comprising the steps of: (a) measuring, before an administration of plasmapheresis, a level of exogenous particles in a blood sample of the individual, thereby generating a pre-treatment level of exogenous particles in the individual; (b) withdrawing a volume of whole blood from the individual; (c) separating the volume of whole blood into a cellular fraction and an exogenous particle fraction, wherein the exogenous particle fraction comprises a level of exogenous particles; and (d) returning the cellular fraction and an exchange fluid to a circulatory system of the individual, (e) measuring, following step (d), a level of particles in a blood sample of the individual, thereby generating a post-treatment level of exogenous particles in the individual; thereby reducing the pre-treatment level of exogenous particles to the post-treatment level of exogenous particles in the individual.

In some aspects, the method further comprises determining a quantitative decrease of the post-treatment level of exogenous particles in the individual compared with the pre-treatment level of exogenous particles in the individual.

In various aspects, the present disclosure provides a method of reducing a level of particles in an individual by plasmapheresis, the method comprising: (a) withdrawing a volume of whole blood from the individual with a pre-treatment level of particles; (b) separating the volume of whole blood into a cellular fraction and a particle fraction, wherein the particle fraction comprises a level of particles; and (c) returning the cellular fraction and an exchange fluid to a circulatory system of the individual, thereby reducing the level of particles in the individual to a post-treatment level of particles.

In some aspects, the particles are exogenous particles.

In various aspects, the present disclosure provides a method of reducing a level of exogenous particles in an individual by plasmapheresis, the method comprising: (a) withdrawing a volume of whole blood from the individual with a pre-treatment level of exogenous particles; (b) separating the volume of whole blood into a cellular fraction and an exogenous particle fraction, wherein the exogenous particle fraction comprises a level of exogenous particles; and (c) returning the cellular fraction and an exchange fluid to a circulatory system of the individual, thereby reducing the level of exogenous particles in the individual to a post-treatment level of exogenous particles.

In various aspects, the present disclosure provides a method of reducing a level of exogenous particles in an individual by plasmapheresis, the method comprising: (a) measuring a pre-treatment level of exogenous particles in a blood sample of the individual prior to performing plasmapheresis; (b) withdrawing a volume of whole blood from the individual; (c) separating the volume of whole blood into a cellular fraction and an exogenous particle fraction, wherein the exogenous particle fraction comprises a level of exogenous particles; and (d) returning the cellular fraction and an exchange fluid to a circulatory system of the individual, thereby reducing the pre-treatment level of exogenous particles to a post-treatment level of exogenous particles in the individual.

In some aspects, the method further comprises e) repeating: steps (b) through (d); steps (a) through (c); steps (a) through (d); or any combination thereof, until the quantitative difference between the post-treatment level of exogenous particles in the individual and the pre-treatment level of exogenous particles in the individual is a target value.

In some aspects, the pre-treatment level of exogenous particles or the pre-treatment level of particles is between 10 to 150 particles, between 20 to 150 particles, or between 30 to 150 particles. In some aspects, the pre-treatment level of exogenous particles or the pre-treatment level of particles is greater than 15, 20, 30, 35, 40, 45, or 50 plastic particles. In some aspects, the pre-treatment level of exogenous particles or the pre-treatment level of particles is greater than 30 plastic particles.

In some aspects, the method further comprises repeating: steps (b) through (d); steps (a) through (c); steps (a) through (d); steps (a) through (e); or any combination thereof, until the post-treatment level of exogenous particles or the post-treatment level of particles is below a threshold level in one or more of the following: the circulatory system of the individual; the organ of the individual; or the blood vessel wall of the individual.

In some aspects, the method further comprises measuring any one of the pre-treatment level of exogenous particles, the pre-treatment level of particles, the post-treatment level of exogenous particles, or the post-treatment level of particles in the circulatory system of the individual. In some aspects, the method further comprises measuring any one of the pre-treatment level of exogenous particles, the pre-treatment level of particles, the post-treatment level of exogenous particles, or the post-treatment level of particles in an organ of the individual. In some aspects, the method further comprises any one of the pre-treatment level of exogenous particles, the pre-treatment level of particles, the post-treatment level of exogenous particles, or the post-treatment level of particles in a blood vessel of the individual.

In some aspects, the method further comprises reducing the post-treatment level of exogenous particles or the post-treatment level of particles to a target value. In some aspects, the target level is less than 100, 200, 300, 400, or 500 particles/μL in the circulatory system of the individual. In some aspects, the target level is between 10-500, 50-100, or 100 and 500 particles/μL in the circulatory system of the individual. In some aspects, the post-treatment level of exogenous particles or the post-treatment level of particles is decreased by from about 10% to about 30%, about 20% to about 40%, about 30% to about 50%, about 40% to about 60%, about 50% to about 70%, or about 40% to about 80% as compared to the pre-treatment level of exogenous particles or the pre-treatment level of particles. In some aspects, the post-treatment level of exogenous particles or the post-treatment level of particles is decreased by from about 30% to about 60% as compared to the pre-treatment level of exogenous particles or the pre-treatment level of particles. In some aspects, the post-treatment level of exogenous particles or the post-treatment level of particles is decreased by up to 10%, 15%, 20%, 25%, 30%, 35%, 40%, 45%, 50%, 55%, 60%, 65%, 70%, 75%, 80%, 85%, or 90% as compared to the pre-treatment level of exogenous particles or the pre-treatment level of particles. In some aspects, the post-treatment level of exogenous particles or the post-treatment level of particles is decreased by at least 10%, 15%, 20%, 25%, 30%, 35%, 40%, 45%, 50%, 55%, 60%, 65%, 70%, 75%, 80%, 85%, or 90% as compared to the pre-treatment level of exogenous particles or the pre-treatment level of particles. In some aspects, the post-treatment level of exogenous particles or the post-treatment level of particles is decreased by at least 40% as compared to the pre-treatment level of exogenous particles or the pre-treatment level of particles.

In some aspects, the method comprises administering of the plasmapheresis comprises exchanging at least one unit of plasma volume. In some aspects, the method comprises administering the plasmapheresis over a plurality of treatment sessions. In some aspects, the method comprises administering the plasmapheresis over a single treatment session. In some aspects, the method comprises administering intravenous immunoglobulin to the individual. In some aspects, the method comprises administering intravenous immunoglobulin to the individual following plasmapheresis. In some aspects, the method comprises administering intravenous immunoglobulin to the individual during the same treatment session as the plasmapheresis. In some aspects, the method comprises administering intravenous immunoglobulin to the individual within 24 hours of the plasmapheresis. In some aspects, the intravenous immunoglobulin is administered at a dose of 2 grams per individual.

In some aspects, the method further comprises performing the method on the individual at least two times per month for at least three months, wherein two of the at least two times per month are within the same week of the month. In some aspects, the intravenous immunoglobulin is administered to the individual at least two times per month.

In some aspects, the exogenous particles or the particles is an inorganic particle or an organic particle. In some aspects, the exogenous particles or the particles are plastic particles. In some aspects, the organic particle is a polymer particle, a carbon particle, or a combination thereof. In some aspects, the polymer comprises plastic, polylactic-co-glycolic acid (PLGA), polyacrylonitrile, polystyrene, polyethylene, low-density polyethylene, high-density polyethylene, polypropylene, polyvinyl chloride, polyurethane, and/or polyethylene terephthalate, poly(l-aspartic acid-co-lactic acid), polyethylene glycol, poly(beta-amino ester), polybutyl cyanoacrylate, or chitosan. In some aspects, the carbon comprises carbon black, graphene oxide, a graphene platelet, a fullerine, a single-walled carbon nanotube, a polycyclic aromatic hydrocarbon, a lipid nanoparticle, or a multi-walled carbon nanotube. In some aspects, the exogenous particles or the particles comprises particles sized: between 1 nm and 2000 nm; between 1 nm and 1000 nm; between 1 nm and 500 nm; or between 1000 nm and 5 mm.

In some aspects, the measuring comprises a spectroscopy method. In some aspects, the measuring comprises a computerized tomography scan, an ultrasound, a positron emission tomography scan, or a magnetic resonance imaging scan. In some aspects, the measuring comprises flow cytometry, near-infrared spectroscopy (NIR), double shot pyrolysis-gas chromatography/mass spectrometry, Fourier transform infrared (FT-IR) spectrometry, visual inspection with an optical microscope Raman spectroscopy, or surface-enhanced Raman scattering, dynamic light scattering (DLS), or surface plasmon resonance. In some aspects, the measuring comprises flow cytometry.

In some aspects, the method further comprises analyzing the exogenous particles or the particles to determine a composition, a size, a distribution of compositions, and/or a distribution of sizes of the exogenous particles or the particles. In some aspects, the exogenous particles or the particles comprise one or more of a nanobead, a nanofiber, a nanosphere, and/or a nanofragment plastic particle. In some aspects, the exogenous particles or the particles comprises one or more of a microbead, a microfiber, and/or a microfragment particle.

In some aspects, the method further comprises reducing a risk of depositing a level of the exogenous particles or the particles onto a surface of a blood vessel or in an organ of the individual. In some aspects, the method further comprises reducing a rate of deposition of the exogenous particles or the particles on a blood vessel or organ of an individual.

In some aspects, the separating comprises centrifugating the volume of whole blood, passing the volume of whole blood through a semipermeable membrane, or a combination thereof. In some aspects, the semipermeable membrane comprises pores of between 0.3 μm and 1.5 μm diameter. In some aspects, the semipermeable membrane comprises pores of between 0.3 μm and 1.0 μm diameter. In some aspects, the semipermeable membrane comprises pores of about 0.3 μm to about 0.5 μm diameter. In some aspects, the semipermeable membrane comprises hollow tubes or fibers.

In some aspects, the exchange fluid comprises one or more therapeutics. In some aspects, the exchange fluid comprises human serum albumin. In some aspects, wherein the exchange fluid comprises 5% of the human serum albumin. In some aspects, the exchange fluid comprises calcium. In some aspects, the exchange fluid comprises Normal Saline. In some aspects, the exchange fluid further comprises intravenous immunoglobulin. In some aspects, the exchange fluid comprises 2 g of the intravenous immunoglobulin.

In various aspects, the present disclosure provides a method of treating a condition in an individual in need thereof comprising performing the method as described herein on the individual, thereby treating the condition.

In some aspects, the condition is associated with exposure to exogenous particles or particles. In some aspects, the condition is a microplastics burden. In some aspects, the microplastics burden is an inflammatory condition, an autoimmune condition, an endocrine condition, a cardiovascular condition, a reproductive condition, or a neurocognitive condition. In some aspects, the microplastics burden is associated with deposition of the exogenous particles on a tissue, organ, or cell of the individual. In some aspects, the tissue is a nerve, vascular, or testicular tissue. In some aspects, the tissue is a cranial nerve tissue. In some aspects, the condition is associated with systemic inflammation or localized inflammation. In some aspects, the localized inflammation is a result of the exogenous particles accumulation at a localized area of inflammation. In some aspects, the localized area of inflammation is a tissue, organ, or cell of the individual. In some aspects, the tissue is a nerve, vascular, or testicular tissue. In some aspects, the condition is an inflammatory, an autoimmune, an endocrine, a cardiovascular, a neurocognitive, or a reproductive disorder.

The present disclosure relates to methods and systems for detecting and removing contaminants from biological fluids and, more particularly, to methods and systems for detecting and removing plastic particles from blood using plasmapheresis.

In various aspects, the present disclosure provides a method of reducing a level of particles in an individual by plasmapheresis, the method including: (a) withdrawing a volume of whole blood from the individual; (b) separating the volume of whole blood into a cellular fraction and a particle fraction, wherein the particle fraction comprises a level of particles; and (c) returning the cellular fraction and an exchange fluid to the circulatory system of the individual, thereby reducing the level of particles in the individual. In some aspects, the particle is an inorganic particle or an organic particle. In some aspects, the particles are plastic particles and the particle fraction is a plastic particle fraction comprising a plurality of plastic particles. In some aspects, the method further comprises reducing a level of particles in the circulatory system of the individual. In some aspects, the method further includes reducing a level of particles in an organ of the individual. In some aspects, the method further includes reducing a level of particles on a blood vessel wall of the individual.

In some aspects, the inorganic particle includes bismuth oxide (Bi₂O₃), silicon dioxide (SiO₂), copper oxide (CuO), zinc oxide (ZnO), titanium dioxide (TiO₂), silver (Ag), gold (Au), platinum (Pt), iron oxide (Fe₂O₃), cerium oxide (CeO₂), cobalt oxide (Co₃O₄), aluminum oxide (Al₂O₃), molybdenum trioxide (MoO₃), magnesium oxide (MgO), nickel oxide (NiO), chromium oxide (Cr₂O₃), tungsten oxide (WO₃), yttrium oxide (Y₂O₃), terbium-doped gadolinium, gold, silver, platinum, nitrate, or manganese oxide (Mn₂O₃). In some aspects, the organic particle is a polymer particle, a carbon particle, or a combination thereof. In some aspects, the polymer comprises plastic, polylactic-co-glycolic acid (PLGA), polyacrylonitrile, polystyrene, polyethylene, low-density polyethylene, high-density polyethylene, polypropylene, polystyrene, polyvinyl chloride, polyurethane, and/or polyethylene terephthalate, poly (l-aspartic acid-co-lactic acid), polyethylene glycol, poly (beta-amino ester), polybutyl cyanoacrylate, polyethylene terephthalate, or chitosan. In some aspects, the carbon particle includes carbon black, graphene oxide, a graphene platelet, a fullerene, a single-walled carbon nanotube, a polycyclic aromatic hydrocarbon, a lipid nanoparticle, or a multi-walled carbon nanotube.

In some aspects, the level of particles includes particles sized: between 1 nm and 2000 nm, between 1 nm and 1000 nm; between 1 nm and 500 nm; or between 1000 nm and 5 mm.

In some aspects, the method includes repeating steps (a) through (c) until the level of particles in one or more of the following is below a threshold level: the circulatory system of the individual; the organ of the individual; and/or the blood vessel wall of the individual. In some aspects, the method includes measuring a level of particles in the circulatory system of the individual. In some aspects, the method includes measuring a level of particles in the organ of the individual. In some aspects, the method includes measuring a level of particles in the blood vessel of the individual.

In some aspects, the measuring is performed before withdrawing the volume of whole blood to yield a pretreatment level of particles. In some aspects, the measuring is performed after returning the cellular fraction and the exchange fluid to the circulatory system of the individual to yield a post-treatment level of particles.

In some aspects, the measuring is performed in real-time during one or more of steps (a) through (c). In some aspects, the method further includes repeating steps (a) through (c) a plurality of times. In some aspects, the method includes repeating steps (a) through (c) until the level of particles in the individual reaches a target level. In some aspects, the target level is not greater than 500 particles/μL in the circulatory system of the individual. In some aspects, the target level is between 100 and 500 particles/μL in the circulatory system of the individual.

In some aspects, the target level is 90%, 80%, 70%, 60%, 50%, 40%, 30%, 20%, 10%, or 1% of the pretreatment level.

In some aspects, the measuring is performed by an imaging method. In some aspects, the measuring is performed using a computerized tomography scan, an ultrasound, a positron emission tomography scan, or a magnetic resonance imaging scan. In some aspects, the measuring is performed using flow cytometry, near-infrared spectroscopy (NIR), double shot pyrolysis-gas chromatography/mass spectrometry, Fourier transform infrared (FT-IR) spectrometry, visual inspection with an optical microscope Faman spectroscopy, or surface-enhanced Raman scattering, dynamic light scattering (DLS), or surface plasmon resonance. In some aspects, the measuring is performed using flow cytometry.

In some aspects, the particle is a plastic particle, and the measuring includes contacting the particle with a fluorescent dye. In some aspects, the fluorescent dye is a lipophilic dye or a phenoxazone dye. In some aspects, the fluorescent dye is Nile red, BODIPY™ 493/503, rhodamine b, or a combination thereof. In some aspects, the measuring includes using an antibody specific for a particle.

In some aspects, the exchange fluid includes an immunoglobulin. In some aspects, the immunoglobulin includes IVIG. In some aspects, the IVIG has a concentration of between 0.5 g/kg and 2 g/kg of bodyweight of the individual.

In some aspects, the method further includes repeating the method once per week, twice per week, three times a week, twice a week, weekly, biweekly, or monthly. In some aspects, the method is repeated over a period of at least one month, at least two months, at least three months, at least four months, at least five months, at least six months, or greater than six months. In some aspects, the method includes analyzing the level of particles to determine a composition, a size, a distribution of compositions, and/or a distribution of sizes of the particles in the level of particles.

In some aspects, the level of particles includes one or more of a nanobead, a nanofiber, a nanosphere, and/or a nanofragment plastic particle. In some aspects, the level of particles includes one or more of a microbead, a microfiber, and/or a microfragment particle.

In some aspects, the method further includes reducing a risk of depositing a level of particles onto a surface of a blood vessel or in an organ of the individual. In some aspects, the method includes reducing a rate of deposition of particles on a blood vessel or organ of an individual.

In some aspects, the separating includes centrifugating the volume of whole blood, passing the volume of whole blood through a semipermeable membrane, or a combination thereof. In some aspects, the semipermeable membrane includes pores of between 0.3 μm and 1.5 μm diameter. In some aspects, the semipermeable membrane comprises pores of between 0.3 μm and 1.0 μm diameter. In some aspects, the semipermeable membrane includes pores of about 0.3 μm to about 0.5 μm diameter. In some aspects, the semipermeable membrane includes hollow tubes or fibers.

In various aspects, the measuring comprises binding a particle with an antibody complex comprising a detectable agent. In some aspects, the measuring includes binding a particle with an aptamer complex comprising a detectable agent. In some aspects, the detectable agent is a fluorescent dye.

In various aspects, the present disclosure provides a system for reducing a level of particles in an individual, the system including: a plasmapheresis unit configured to separate particles from whole blood; and a measuring unit configured to analyze a sample of particles. In some aspects, the system is configured to perform the method previously described. In some aspects, the system further includes a processor for operating the plasmapheresis unit and/or measuring unit. In some aspects, the measuring unit is a computer tomography scanner, an ultrasound machine, a magnetic resonance imaging scanner, a flow cytometer, an immunoassay, a mass spectrometer, a Raman spectrometer, a surface plasmon resonance instrument, or a Fourier Transform Infrared (FT-IR) spectrometer. In some aspects, the measuring unit is a flow cytometer. In some aspects, the flow cytometer comprises a violet side scatter unit.

In various aspects, the system includes a display. In some aspects, the display is configured to show real-time particle levels in withdrawn whole blood. In some aspects, the processor is further configured to depict a particle level, a target particle level, or a combination thereof. In some aspects, the system is automated. In some aspects, the system further includes blood collection tubes comprising polyethylene terephthalate. In some aspects, the system further includes a centrifugal blood separation unit. In some aspects, the system further includes a semipermeable membrane. In some aspects, the system further includes a coating configured to adsorb a particle. In some aspects, the coating comprises a polystyrene sulfonate or a polyaspartate. In some aspects, the system further includes a plastic-specific adsorption column.

### BRIEF DESCRIPTION OF THE DRAWINGS

These and other features, embodiments, and advantages of the present invention will become better understood with regard to the following description, and accompanying drawings where:

FIG. 1A is a schematic diagram showing therapeutic plasma exchange (TPE) treatment. The figure provides a study design with two experimental groups. One experimental group receives TPE without the addition of intravenous immunoglobulin (IVIG) and one experimental groups receives TPE with the addition of IVIG.

FIG. 1B is a schematic diagram showing the timing to therapeutic plasma exchange (TPE) therapy sessions for an experimental regimen. The figure provides exemplary time points for TPE sessions (may comprise an actual TPE therapy session or a Sham session as an experimental control) and sample collection. As shown in the left-hand side of FIG. 1B, an exemplary 3-month experimental regimen may comprise TPE therapy (or a control Sham therapy) at the 0, 1, and 2-month marks. As shown in the figure, the TPE therapy (or a control Sham therapy) sessions may be administered as two sessions close together at the 0, 1, and 2-month marks with sample collections at the 0, 1, and 2-month marks. As shown on the right-hand side of FIG. 1B, an exemplary 6-month experimental regimen may comprise TPE therapy (or a control Sham therapy) at the 0, 1, 2, 3, 4, and 5-month marks. As shown in the figure, the TPE therapy (or a control Sham therapy) sessions may be administered as a single session at the 0, 1, 2, 3, 4, and 5-month marks with sample collections at the 0, 3, and 5-month marks.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary embodiments described herein.

The presence of micro- and nanoparticles in the human body can pose health risks due to their small size, allowing them to penetrate biological barriers and accumulate in various tissues and organs. Moreover, micro- and nanoparticles can carry and release toxic substances, leading to inflammation, oxidative stress, and other adverse health effects. Therefore, there is a need for effective methods to detect and remove these tiny particles from the human body.

Particles refer to tiny particles that are less than 5 millimeters in size. Particles less than 1000 nanometers (nm) in size are generally referred to herein as nanoparticles and, in some embodiments, can be as small as 1 nm. Larger particles, between about 1000 nm to 5 mm, are referred to herein as microparticles. These particles can originate from a variety of sources, including the degradation of larger materials in the environment, and can be composed of various types of materials. Micro- and nanoparticles are ingested or inhaled by living organisms and accumulate in various tissues and organs. There is growing concern that these particles may cause adverse effects such as inflammation and oxidative stress. Additionally, micro-and nanoparticles can act as carriers for other pollutants, potentially increasing the toxicity of these contaminants. There is a need for effective methods to quantify and remove these particles from the human body, to reduce their detrimental health effects.

Current methods for detecting and removing micro-and nanoparticles in biological samples face several challenges, including the small size of the particles and the complexity of biological matrices. Traditional techniques such as microscopy and spectroscopy have limitations in terms of sensitivity and specificity when applied to micro- and nanoparticle detection in complex biological samples. The present disclosure presents effective, rapid, and safe methods of detecting and removing particles. The disclosed techniques for detecting and removing particles from the bloodstream can reduce the environmental and healthcare burdens faced by modern society.

### Overview of Treatment Modalities

The term “plasmapheresis” as used herein is interchangeable with the term “therapeutic plasma exchange.” Plasmapheresis is a form of apheresis wherein some amount of the plasma of an individual is withdrawn from the body of the individual and removed. In the present disclosure, the plasma includes plastic particles. Plasmapheresis methods are described herein for detecting and removing plastic particles. In removing plastic particles, the plasmapheresis methods described herein may also treat diseases or disorders associated with plastic particle accumulation in blood and tissue.

A plasmapheresis treatment for removing plastics includes—and begins with—the withdrawing of whole blood, or peripheral blood (PB) from an individual receiving the plasmapheresis treatment. Whole blood, or peripheral blood are withdrawn is separated into a cellular fraction and a fraction including plastic particles. The term “cellular fraction” as used herein can refer to or comprise red blood cells, white blood cells, and platelets. The terms “particle fraction” as used herein can refer to or include a liquid portion of whole blood, or peripheral blood, which contains, among other things, proteins, electrolytes, vitamins, hormones, and particles. In the plasmapheresis treatment disclosed herein, a particle fraction is removed while a cellular fraction is returned to the individual receiving the plasmapheresis treatment. As used herein in the context of plasmapheresis administration (or any other type of apheresis procedure), the terms “withdraw,” “withdrawal,” “withdrawn,” and “withdrawing” (or any other conjugation of “withdraw”) means to draw blood out (actively or passively) from the vascular system of an individual receiving plasmapheresis (or other type of apheresis procedure) which may be achieved using any suitable vascular access, which includes but is not limited to peripheral intravenous lines and midline or central lines. As used herein in the context of plasmapheresis administration (or any other type of apheresis procedure), the terms “return” or “returning” (or any other conjugation of “return”) or “infuse,” or “infusing” (or any other conjugation of “infuse”) means to return blood or an exchange fluid back (actively or passively) to the vascular system of an individual receiving plasmapheresis (or other type of apheresis procedure) which may be achieved using any suitable vascular access. As used herein in the context of plasmapheresis administration (or any other type of apheresis procedure), the terms “remove,” “removal,” “removed,” and “removing” (or any other conjugations of “remove”) means to remove at least a portion of whole blood, or peripheral blood, withdrawn from an individual receiving plasmapheresis (or any other apheresis procedure) and not returning the portion of the whole blood, or peripheral blood, to the individual receiving plasmapheresis so that it is removed from their body. As used herein, in the context of plasmapheresis (or any other apheresis procedure), the terms “separate,” “separated,” or “separating” (or any other conjugation of “separate”) means to separate apart components of blood from one another. For example, in plasmapheresis, whole blood or peripheral blood (PB) are withdrawn, and a particle fraction is separated from the cellular fraction of the withdrawn whole blood or peripheral blood. As used herein, the term “plasmapheresis” may be combined with other terms such as “therapy” (i.e., plasmapheresis therapy) or “treatment” (i.e., plasmapheresis treatment) or “procedure” (i.e., plasmapheresis procedure) and, unless otherwise indicated, no specific meaning should be attributed to the use of one of these terms or the other in the context in which they appear. Of note, the term “plasmapheresis” is often used interchangeably with therapeutic plasma exchange, and at other times it is used to denote a form of therapeutic plasma exchange where less plasma is removed than that removed in therapeutic plasma exchange. To avoid confusion with respect to terminology, the term plasmapheresis is used throughout, and as stated, as used herein, the term therapeutic plasma exchange is interchangeable with the term plasmapheresis. However, in no way should the term plasmapheresis be deemed to be limiting on the scope of the disclosure found herein which may be relevant to different types of apheresis based on context.

A plasmapheresis therapy session may begin with the initial step of withdrawing whole blood, or peripheral blood from a blood vessel of a patient using an apheresis device. Apheresis devices are machines configured to carry out procedures, including plasmapheresis. Apheresis devices may be configured to withdraw whole blood, or peripheral blood from an individual through an intravenous line, separate the whole blood, or peripheral blood into components, and return an infusion to the individual through an intravenous line. The infusion returned to the individual may include separate components which may include blood that has had the plasma component removed from it. In addition, an infusion given to the individual may also include an exchange fluid. An apheresis device can be an ex vivo apheresis system or machine comprising one or more centrifugal chambers. An ex vivo apheresis system or machine can also comprise a return flow controller and one or more sensors for monitoring plasma or blood density. An apheresis device can also be configured to deliver an anticoagulant to the patient during the procedure. In some embodiments, the anticoagulant can be citrate dextrose or heparin. It should, however, be understood that any method or device for carrying out plasmapheresis is suitable for use with the methods and formulations described herein, and the description provided should not be deemed to limit the inventive methods or formulations described herein which are suitable for use with any device or method for carrying out plasmapheresis.

The present disclosure also involves the collection of human blood or plasma samples for particles (e.g., exogenous particles) analysis. These samples may be fresh, frozen, liquid, or dried samples. In some embodiments, the samples are collected at a volume from 0.5 to 2 milliliters for particles (e.g., exogenous particles analysis). The collected samples may be subjected to a comprehensive digestion protocol designed to remove or degrade biological macromolecules such as proteins, lipids, nucleic acids, and cellular debris, which otherwise may interfere with subsequent staining or detection steps of particles (e.g., exogenous particles).

The volume of plasma removed is suitable for removing particles from the individual and can be between about 0.5 to 2 times the plasma volume of the patient. The plasmapheresis is repeated at intervals suitable for removing micro-or nanoparticles from blood, organs, or a combination thereof. The plasmapheresis can be repeated at least one time a week or several times a month depending on the level of particles, the types of particles, the tissues targeted for particle removal, or the desired target, reference, or baseline level of particles. The plasmapheresis is performed over a period of 1, 2, 3, 4, 5, 6 or more months.

In some embodiments, therapeutic plasma exchange (TPE) may comprise a procedure in which the patient's blood is passed through an apheresis machine, wherein the filtered plasma may be removed and discarded and then the collected red blood cells, white blood cells and platelets may be combined with replacement fluid, such as plasma, Normal Saline, or albumin, which is then delivered back into the patient (e.g., into the patient's circulatory system). TPE may be employed in the treatment of various autoimmune and neurological disorders, such as Guillain-Barré syndrome, myasthenia gravis, thrombotic thrombocytopenia purpura (TTP), and certain types of vasculitis.

A TPE procedure may comprise inserting a catheter into a vein, through which blood is withdrawn and circulated through a blood separation device (e.g., an apheresis device). The blood separation device may separate plasma from the blood cells using centrifugation or filtration. In some embodiments, the blood separation device is an apheresis device. The blood cells (e.g., the red blood cells, white blood cells, platelets, or any combination thereof) may then be recombined with a replacement fluid and returned to the patient's bloodstream. A single session of TPE may last between one and three hours, and the number of sessions required may depend on the specific condition being treated and the patient's response. Overall, TPE may be a valuable tool in the management of various immune-mediated conditions, especially when rapid removal of circulating pathogenic factors is essential. TPE may significantly improve the outcomes of various medical indications.

### Methods for Removing Particles by Plasmapheresis

### Types of Particles

The methods described herein are directed to reducing a level of particles in an individual. Tiny particles can become problematic when ingested, inhaled, or absorbed in other ways. The particle can be a bead, a fiber, a sphere, a fragment, or an irregular particle. In the case of a nanoparticle, the particle can be a nanobead, a nanofiber, a nanosphere, a nanofragment, or a nanoparticle. In the case of a microparticle, the particle can be a microbead, a microfiber, a microsphere, a microfragment, or a microparticle. In some embodiments, the particle is a nanoplastic. In other embodiments, the particle is a microplastic. A particle such as a nanofiber or microfiber can have a high aspect ratio with a length of at least 200 times the diameter, and has a large surface area relative to the volume.

The size of a particle can be from about 1 nm to about 1000 nm in size, and the particle is referred to herein as a “nanoparticle.” The size of a nanoparticle can also be between about 1 nm to about 500 nm, between 1 nm to 200 nm, between 1 nm to 500 nm, or between 1 nm to 1000 nm. A nanoparticle can also be between 1 nm and 2000 nm. A particle with a size from about 1000 nm to about 5 mm is referred to herein as a “microparticle.” A macroparticle can be between 5 mm to 10 mm in size or greater. In some embodiments, the size of a microparticle is between about 1000 nm to about 1 mm, between about 1000 to about 2 mm, or between about 1000 nm to about 5 mm.

Such particles can be inorganic or organic and can be generated from smoke, manufacturing, emissions, household activities, fires, dust storms, volcanoes, power generation, agriculture, lightning, sewage treatment, or other sources. Inorganic particles can be metals, metalloids, or phosphors. For example, inorganic particles can include bismuth oxide (Bi₂O₃), silicon dioxide (SiO₂), copper oxide (CuO), zinc oxide (ZnO), titanium dioxide (TiO₂), silver (Ag), gold (Au), nitrate, platinum (Pt), iron oxide (Fe₂O₃), cerium oxide (CeO₂), cobalt oxide (Co₃O₄), aluminum oxide (Al₂O₃), molybdenum trioxide (MoO₃), magnesium oxide (MgO), nickel oxide (NiO), chromium oxide (Cr₂O₃), tungsten oxide (WO₃), yttrium oxide (Y₂O₃), terbium-doped gadolinium, gold, silver, platinum, or manganese oxide (Mn₂O₃).

Organic particles contain carbon and can be in many different forms. Organic particles can be polymer particles, carbon particles, or a combination thereof. Plastic is a type of polymer; however, other polymers can be particles as well and can contaminate the blood, tissue, and organs of an individual. Types of polymer particles include polylactic-co-glycolic acid (PLGA), polyacrylonitrile, polystyrene, polyethylene, low-density polyethylene, high-density polyethylene, polypropylene, polyvinyl chloride, polyurethane, and/or polyethylene terephthalate, poly (l-aspartic acid-co-lactic acid), polyethylene glycol, poly (beta-amino ester), polybutyl cyanoacrylate, or chitosan, among others. Carbon particles can be somewhat different from the previously described polymer particles and can include carbon black, graphene, graphene oxide, a graphene platelet, a fullerene, a single-walled carbon nanotube, a polycyclic aromatic hydrocarbon, a multi-walled carbon nanotube, lipid nanoparticles, or other carbon compounds.

Types of plastic particles can include acrylonitrile, acrylonitrile butadiene styrene, polyacrylonitrile, polycarbonate, polyethylene, low-density polyethylene, high-density polyethylene, crystalline polyethylene, polypropylene, polystyrene, polyvinylchloride, polyethylene terephthalate, polyurethanes, or other materials. Types of plastic particles removed using the present methods also include fibers such as polyester fibers, polyamide fibers, and acrylic fibers. The plastic particles can further include epoxide resin, phenol-formaldehyde resin, or unsaturated polyester resin. Additives can also be included in or on plastic particles, such as plasticizers, contaminants, ultraviolet stabilizers, or flame retardants.

Reducing the level of particles in an individual can include reducing the level of particles in the bloodstream or adhering to a blood vessel, organ, or tissue of the individual. The methods also include reducing the risk of depositing a level of particles onto the surface of a blood vessel or organ of the individual. In some embodiments, the methods include reducing the rate of deposition of particles on a blood vessel or organ of an individual. In some embodiments, the particles are plastic particles.

### Plasmapheresis Treatment—Particle Measurements

In an exemplary method, a plasmapheresis method can begin with the step of identifying an individual in need of a plasmapheresis therapy. A blood sample is taken from the individual, either before initiation of the plasmapheresis or from the whole blood, or peripheral blood, that is withdrawn, and used to determine a level of particles described herein.

The methods disclosed herein may include analyzing a blood sample, the cellular fraction, and/or the fraction containing particles to quantify a level of particles, type of particles, size of particles, or a combination thereof. This analysis can provide information about the amount, type, and size of particles present in the individual's body and the effectiveness of the plasmapheresis in removing these particles. The analysis can be performed using various techniques, such as through a computed tomography (CT) scan, an ultrasound, a magnetic resonance imaging (MRI) scan, a positron emission tomography (PET) scan, an immunoassay, flow cytometry, double shot pyrolysis—gas chromatography/mass spectrometry, dynamic light scattering (DLS), Fourier-transform infrared spectroscopy (FTIR), laser infrared imaging spectrometry (LDIR), near-infrared spectroscopy (NIR), surface plasmon resonance (SPR), visual inspection with an optical microscope, Raman spectroscopy, or surface-enhanced Raman scattering. The analysis can be performed several hours to several days or weeks prior to performing the method, immediately prior to performing the method, during the method in real-time, and/or after performing the method.

Components used during the analysis, plasmapheresis, removal of particles, and replacement of plasma with exchange fluid can be cleaned and monitored to avoid contamination of equipment and chemicals used with particles. Hoses, tubes, liquids, and beakers can be regularly cleaned and tested for particle contamination.

The measurement of a level of particles may include obtaining a blood sample from the individual immediately before performing plasmapheresis. This pre-plasmapheresis blood sample can serve as a baseline for comparison with the particle fraction obtained during plasmapheresis. By comparing the amount, type, or size of particles in the pre-plasmapheresis blood sample with those in the removed particle fraction, the effectiveness of the plasmapheresis in removing particles can be assessed.

The particle fraction removed during plasmapheresis can be stored for later analysis of particles. This can allow for a more detailed and comprehensive analysis of the particles, which may not be feasible to perform in real-time during the plasmapheresis. The blood sample may be obtained immediately before, during, or after performing plasmapheresis. The pre-plasmapheresis blood sample can serve as a baseline for comparison with the particle fraction obtained during plasmapheresis.

The measurement of particles can be performed prior to or after plasmapheresis and can also be performed in real-time during the plasmapheresis. This can allow for the continuous monitoring of the particles and can provide immediate feedback on the effectiveness of the plasmapheresis in removing particles. The analysis can detect level, size, and/or type of particles in the whole blood, or in peripheral blood, tissue or organ of an individual.

To measure the level, size and/or type of particles, a sample is taken of the whole blood, peripheral blood, the plasma, the cellular fraction or the particle fraction of an individual, or a sample of a tissue or organ is taken.

The samples may be analyzed for particles (e.g., exogenous particles) using one or more different techniques. Many techniques are developed to analyze blood; however, the detection and analysis of particles may be more complex than other compounds due to the presence of other compounds in blood, such as triglycerides.

The samples may be analyzed for particles (e.g., exogenous particles) by a spectroscopy method, an imaging method, a cytometry method, or any combination thereof. In some embodiments, a sample is analyzed for particles (e.g., exogenous particles) by spectral flow cytometry, a technique that measures the physical and chemical characteristics of particles in a fluid as it passes through at least one laser beam. In some embodiments, a sample is analyzed for particles (e.g., exogenous particles) by incubation with a fluorescent dye, which labels the particles for subsequent detection. The labelled sample of particles may be then passed in front of a laser and the sample may scatter and emit light at varying wavelengths which is then detected and converted into electronic signals, which are processed by a computer to generate and display detailed data about the particles (e.g., quantification, size, distribution, concentration, etc.). When the analysis is performed in real time, the parameters of the method can be changed according to the display; for example, when the level of particles is low compared to a reference, target, or baseline level, the volume of plasma removed can be reduced, or the amount of whole blood, or peripheral blood processed can be reduced, or other variations can be affected that alter the treatment results. When a level of particles is high compared to a reference, target, or baseline level, the volume of plasma removed can be increased, or the amount of whole blood, or peripheral blood processed can be increased, or other variations can be used to increase the effectiveness of the method.

Instrument settings, including laser excitation wavelength and emission detection parameters may be calibrated using standard polymer nanoparticles of known composition and size, such as polystyrene or polyethylene beads ranging from 200 to 1000 nanometers in diameter. These standards serve as both positive controls for assay sensitivity and benchmarks for recovery efficiency throughout the sample processing workflow.

In some embodiments, a sample is analyzed for particles (e.g., exogenous particles) by performed with flow cytometry with a fluorescent dye that may be a lipophilic or phenoxazone dye, and appropriate dyes include Nile Red, BODIPY™ 493/503, or Rhodamine B. The fluorescent dye can also be a commercially available dye, including iDye™ Poly 456 Pink™, iDye™ Poly 451 Blue™, or Rit™ DyeMore Synthetic Kentucky Sky™. Nile Red is a biologically compatible fluorescent dye that binds to several different types of particles, allowing for the detection and quantification of particles in a blood sample with low background noise contamination. It is a red phenoxazone dye that binds to the surface of plastics and neutral lipids.

To label the particles, a blood sample may be incubated with the fluorescent dye for a period ranging from 5 minutes to 2 hours, depending on the dye and sample characteristics. The concentration of the fluorescent dye can be optimized to ensure efficient binding to particles without causing excessive background fluorescence. The incubation may be performed at room temperature or at an elevated temperature, such as 37° C., to enhance dye penetration and binding. Due to possible environmental contamination of reagents, tubing, vials, etc. with particles, reagents and equipment are regularly cleaned and tested for particle contamination. Clean, filtered or treated reagents are used to reduce contamination of testing materials with particles that are not associated with the sample. To further reduce contamination, samples can be manipulated in a biosafety level 2 environment.

In another method of analyzing the sample, the sample can be incubated to degrade organic matter for up to 10 days. Pretreatment includes use of oxidizing agents, alkali digestion, density separation, incubation with a solution such as 1% aqueous potassium hydroxide, or a combination thereof. The sample can be incubated with a fluorescent dye after organic matter is degraded, or without degrading organic matter. The sample can be incubated with a fluorescent dye at concentrations ranging from 0.1 to 10 μg/mL. The dye may be dissolved in various solvents such as acetone, ethanol, or dimethyl sulfoxide (DMSO), depending on the specific dye and sample characteristics. Incubation times may vary from 5 minutes to 2 hours, with temperatures ranging from room temperature (20-25° C.) to 37° C. After incubation, the sample may be diluted with a suitable buffer solution, such as phosphate-buffered saline (PBS) or Tris-buffered saline (TBS), to reduce background fluorescence. In some embodiments, the labeled sample may undergo a washing step to remove excess unbound dye before analysis. In some embodiments, a nanofiber or microfiber can be contacted with higher amounts of dye than a bead, sphere, or fragment with the same diameter, leading to higher detection levels with flow cytometry.

The diluted or un-diluted labeled sample can be analyzed using flow cytometry with excitation and emission wavelengths optimized for the chosen fluorescent dye. Excitation wavelengths may range from about 400 to 650 nm, while emission wavelengths may range from about 400 to 700 nm, depending on the specific dye used. Nile Red can be excited at 561 nm, with emissions collected with a 585/16 bandpass filter. Violet side scatter filters (405 nm) can also be used to increase the ability to detect smaller particles. Appropriate controls can be included in each experiment, such as unlabeled samples and samples spiked with known concentrations of particles, to establish baseline fluorescence and validate the labeling procedure. The specific parameters may be adjusted based on the type of particles being detected, the sample matrix, and the sensitivity requirements of the analysis. Components used are rinsed and cleaned to avoid contamination between runs. For calibration, small beads of known sizes can be used. For detecting levels of plastic particles, the plastic particles can be polyacrylonitrile, polystyrene, polyethylene, low-density polyethylene, high-density polyethylene, polypropylene, polymethyl methacrylate, polyvinyl chloride, polyethylene vinyl acetate, polyethylene terephthalate, other plastics, or a combination thereof.

The labeling protocol may also include additional steps such as sample pre-treatment to remove interfering substances, centrifugation to concentrate particles, or the use of detergents to improve dye penetration. For some applications, the labeling process may be automated using a robotic liquid handling system to ensure consistency and reduce human error. This automated system could be integrated with the flow cytometer for high-throughput analysis.

The labeling protocol may also include additional steps such as sample pre-treatment to remove interfering substances, centrifugation to concentrate particles, or the use of detergents to improve dye penetration. In some embodiments, a fixation step using formaldehyde or glutaraldehyde may be employed to preserve the sample and enhance labeling stability.

Fourier Transform Infrared (FTIR) spectrometry can be used to analyze the sample to detect particles. FTIR spectrometry is a technique used to obtain an infrared spectrum of absorption or emission of a solid, liquid or gas. This technique can be used to identify different types of particles based on their distinct infrared absorption spectra.

Raman spectroscopy can be used to analyze the sample to detect particles. Raman spectroscopy is a spectroscopic technique used to observe vibrational, rotational, and other low-frequency modes in a system. This technique can be used to identify different types of particles based on their distinct Raman spectra. To increase sensitivity of Raman spectroscopy, the sample can be placed on a nanometer grid of gold that amplifies a light signal from the particles.

Surface plasmon resonance (SPR) can be used to analyze the sample to detect particles. Surface plasmon resonance is an optical technique utilized for detecting molecular interactions that occur at the surface of metal films.

Surface plasmon resonance offers several benefits for detecting particles in samples. SPR can provide real-time, quantitative measurements of particle binding interactions with high sensitivity. In an SPR setup for particle detection, a metal film surface is functionalized with antibodies or aptamers specific to materials in the particles; for example, polyethylene or polystyrene. As the sample flows over this functionalized surface, particles bind to the immobilized antibodies/aptamers, causing a measurable loss of intensity and change in refractive index of the reflected beam.

The sensitivity of SPR allows for detection of low levels of particles. Additionally, SPR can provide information on binding kinetics and affinity, which could help characterize the types and properties of particles present in the sample. Multiple channels on an SPR instrument could be used to simultaneously detect different types of particles using various specific antibodies.

However, the complex nature of samples may present challenges for SPR analysis, such as non-specific binding of other blood components to the sensor surface. Careful sample preparation and the use of appropriate controls ensure accurate detection of particles. Despite these challenges, the high sensitivity and real-time capabilities of SPR are useful for analyzing particles in samples, particularly when combined with other analytical methods.

Analyzing the sample to detect particles can include contacting the sample with a substrate conjugated to a known plastic, allowing particles in the sample to compete with the conjugated particle for binding to an antibody or aptamer specific to the type of particle, and detecting the amount of antibody or aptamer bound to the substrate. The substrate can be a magnetic bead or a microfluidic chip. The antibody or aptamer can be labeled with a fluorescent dye. The known particle can be a plastic particle, which in some embodiments can be polyacrylonitrile, polystyrene, polyethylene, low-density polyethylene, high-density polyethylene, polypropylene, polyvinyl chloride, polyethylene terephthalate, or a combination thereof.

The sample can be analyzed to detect particles using an antibody that is specific for a plastic. The antibody can bind to the plastic, allowing for the detection and quantification of particles in the sample.

Analyzing the sample to detect particles can include incubating the sample with a substrate conjugated to an antibody or aptamer specific to a plastic, washing the substrate to remove unbound material, eluting bound particles from the substrate, and analyzing the eluted particles. Analyzing the eluted particles can include a technique including through a computed tomography (CT) scan, an ultrasound, a magnetic resonance imaging (MRI) scan, a positron emission tomography (PET) scan, an immunoassay, flow cytometry, double shot pyrolysis-gas chromatography/mass spectrometry, dynamic light scattering (DLS), Fourier-transform infrared spectroscopy (FTIR), laser infrared imaging spectrometry (LDIR), near-infrared spectroscopy (NIR), surface plasmon resonance (SPR), visual inspection with an optical microscope, Raman spectroscopy, or surface-enhanced Raman scattering.

The methods disclosed herein may include analyzing the sample to detect particles using an antibody or aptamer that is specific for a plastic. The antibody or aptamer can bind to the plastic, allowing for the detection and quantification of particles in the sample. The antibody or aptamer can be labeled with a fluorescent dye, which can emit light when excited by a laser, allowing for the detection of the bound particles using techniques such as flow cytometry. In some embodiments, the antibody or aptamer can be conjugated to a substrate, such as a magnetic bead or a microfluidic chip. The substrate can provide a solid support for the antibody or aptamer, facilitating the binding of the particles and the subsequent detection and quantification of the bound particles.

The methods disclosed herein may include contacting the sample with a substrate conjugated to a known particle. The known particle material can serve as a competitive inhibitor, competing with the particles in the sample for binding to the antibody or aptamer. The amount of antibody or aptamer bound to the substrate can then be detected and used to estimate the amount of particles in the sample. The known particle can be a plastic, and in some embodiments that plastic can be selected from various types of plastic particles, such as polystyrene, polyethylene, polypropylene, polyvinyl chloride, and polyethylene terephthalate.

The methods disclosed herein can include incubating the sample with a substrate conjugated to an antibody or aptamer specific to a particle, washing the substrate to remove unbound material, eluting bound particles from the substrate, and analyzing the eluted particles. The eluted particles can be analyzed using various techniques, such as through a computed tomography (CT) scan, an ultrasound, a magnetic resonance imaging (MRI) scan, a positron emission tomography (PET) scan, an immunoassay, flow cytometry, double shot pyrolysis-gas chromatography/mass spectrometry, dynamic light scattering (DLS), Fourier-transform infrared spectroscopy (FTIR), laser infrared imaging spectrometry (LDIR), near-infrared spectroscopy (NIR), surface plasmon resonance (SPR), visual inspection with an optical microscope, Raman spectroscopy, or surface-enhanced Raman scattering. These techniques can provide detailed information about the amount, type, and size of the particles, contributing to a comprehensive analysis of the particles in the sample. The results of the analysis of the levels and types of particles determine the frequency of administration of plasmapheresis, and can also determine the amount and composition of exchange fluid used.

Alternatively, the individual can be subjected to imaging to measure the particles. The imaging can be a CT scan, an ultrasound, an MRI, or similar imaging techniques.

### Plasmapheresis Treatment·Particle Separation

When the level of particles is above a baseline or target level, the plasmapheresis method is administered. The plasmapheresis method further includes withdrawing whole blood or peripheral blood from a blood vessel of the individual receiving plasmapheresis using an apheresis device or other technique for withdrawing blood. The whole blood or peripheral blood withdrawn is separated into a cellular fraction and a particle fraction using the apheresis device or other technique for separating blood components. Separating the whole blood or peripheral blood into a cellular fraction and a particle fraction can be performed using centrifugation or by exposing the whole blood or peripheral blood to at least one semipermeable membrane, which has pores of a size to allow the particles to pass through while retaining cells and related material. A combination of centrifugation and membrane separation can also be performed. During centrifugation, components of the whole blood or peripheral blood are separated by density, with low-density components, such as plastic particles, remaining towards the middle of the centrifuge. During filtration through a semipermeable membrane, pores in the semipermeable membrane remove smaller components, such as plastic or metal particles, while retaining larger components, such as cells. Additional membranes can further separate different components by size and/or shape depending on the size of the pores in the membranes. When whole blood or peripheral blood is separated using filtration, the same plasma can be delivered back to the patient if needed. When whole blood or peripheral blood is separated by centrifuging, an exchange fluid is returned to the patient instead of the plasma.

A semipermeable membrane comprises pores of about 0.3 μm to about 1 μm in diameter. In some embodiments, the semipermeable membrane comprises pores of about 0.3 μm to about 0.5 μm diameter. The semipermeable membrane can be a hollow tube or fiber.

The method disclosed herein is directed to removing a level of particles from whole blood or peripheral blood, a blood vessel, or an organ of an individual and includes performing plasmapheresis at least one, two, three, four, or more times per week, with at least two of the days being one day apart, or with at least two of the days being two days apart, or with at least two of the treatments being in a single week. The treatments are performed for at least one month, two months, three months, four months, five months, six months, or longer. In some embodiments of the methods described herein, each plasmapheresis treatment session can last approximately 90 minutes to 2 hours. However, it should be understood that a plasmapheresis session length can be varied based on the objective of the plasmapheresis treatment and sessions shorter than 90 minutes or longer than 2 hours are suitable with the methods and formulations described herein. In addition, a plasmapheresis treatment session duration can vary depending on different factors associated with the individual receiving the plasmapheresis, including but not limited to the level of particles in the individual, the weight of the individual receiving the plasmapheresis or the overall health of the individual.

### Exchange Fluid Volumes

The treatment method can further comprise infusing back to the individual receiving plasmapheresis an exchange fluid and the cellular fraction while removing the particle fraction from the individual. The particle fraction also includes plasma; in some embodiments, when the particles are removed from the plasma, the same plasma can be returned to the patient. An exchange fluid can be administered with the plasmapheresis wherein the exchange fluid is administered to the individual receiving plasmapheresis intravascularly (i.e., through intravenous access; e.g., peripheral, midline, or central line) during the plasmapheresis treatment.

Apheresis devices may be configured to withdraw whole blood from an individual through an intravenous line, separate the whole blood into components, and return an infusion to the individual through an intravenous line. The infusion returned to the individual may include separate components which may include blood that has had the plasma component removed from it. In addition, an infusion given to the individual may include an exchange fluid as well. An apheresis device can be an ex vivo apheresis system or machine comprising one or more centrifugal chambers. An ex vivo apheresis system or machine can also comprise a return flow controller and one or more sensors for monitoring plasma or blood density. An apheresis device can also be configured to deliver an anticoagulant to the patient during the procedure. In some embodiments, the anticoagulant can be citrate dextrose. It should, however, be understood that any method or device for carrying out plasmapheresis is suitable for use with the methods and formulations described herein and the description provided should not be deemed to limit the inventive methods or formulations described herein which are suitable for use with any device or method for carrying out plasmapheresis.

An exchange fluid may be administered with plasmapheresis (e.g., therapeutic plasma exchange (TPE)) wherein the exchange fluid is administered to the individual receiving plasmapheresis intravascularly (e.g., through intravenous access of a peripheral, midline, or central line) during the plasmapheresis treatment. An exchange fluid may comprise any fluid that is suitable for use in intravenous fluid administration. For example, non-limiting examples of fluids suitable for intravascular administration with the administration of plasmapheresis as described herein are commonly referred to as isotonic fluids and include Normal Saline (e.g., a 0.9% saline solution) and Lactated Ringerts. An exchange fluid solution suitable for use in plasmapheresis as described herein may further include albumin such as human albumin. For example, an exchange fluid may comprise a normal saline solution that includes 5% albumin by weight. Typically, a source of albumin is human derived albumin also referred to as human serum albumin (HSA). As an example, an exchange fluid suitable for use with plasmapheresis may comprise a sterile liquid preparation comprising an amount of human-derived protein in the amount of 50 g per 1000 ml of the sterile liquid preparation wherein at least 96% of the human-derived protein is human serum albumin protein. Besides the human-derived serum albumin protein, the remainder of the HSA preparation can comprise a saline solution and small amounts of potassium, N-acetyl-DL-tryptophan, caprylic acid, or a combination thereof. A 5% HSA preparation of an exchange fluid can be an FDA-approved 5% HSA preparation. More specifically, the 5% HSA preparation can be manufactured by an FDA-approved procedure such as the Cohn-Oncley cold ethanol fractionation procedure followed by ultra-filtration and pasteurization. Replacing plasma withdrawn from an individual receiving plasmapheresis with 5% HSA is useful for regulating and stabilizing the volume of circulatory blood within the individual. In some embodiments, the exchange fluid may comprise 5% HSA in Normal Saline. In some embodiments, the exchange fluid may comprise 5% HSA and calcium in Normal Saline. In some embodiments, the exchange fluid may comprise 5% HSA, calcium, and IVIG (e.g., 2 g IVIG) in Normal Saline. In some embodiments, the exchange fluid may comprise 5% HSA and IVIG (e.g., 2 g IVIG) in Normal Saline.

An exchange fluid may be mixed or combined in any suitable way with the cellular fraction of the whole blood that is withdrawn during plasmapheresis, which remains after separation of plasma and which is returned to the individual receiving plasmapheresis during the procedure. An exchange fluid suitable for use with plasmapheresis may also include one or more therapeutics. For example, a therapeutic that reduces inflammation may be provided with plasmapheresis by, for example, mixing the therapeutic with an exchange fluid. An exchange fluid for plasmapheresis may also comprise or be mixed together one or more blood products (i.e. not including the cellular fraction that is returned) including but not limited to fresh frozen plasma and platelets. It should be understood, that when mixed with an exchange fluid, therapeutics and blood products will to at least some degree be withdrawn back from the individual receiving the plasmapheresis and as such it may be beneficial to administer or infuse a therapeutic and/or blood product after completing the blood withdrawal from the individual so that the therapeutic and/or blood product will not be withdrawn from the individual during the plasmapheresis therapy.

A volume of exchange fluid returned to the individual may be approximately equal to a volume of plasma that is removed. For example, if one plasma volume is removed from an individual receiving plasmapheresis, in the methods described herein, the volume of an exchange fluid returned to the individual will also be approximately equal to one plasma volume. Alternatively, the volume of exchange fluid returned may exceed the amount of plasma volume removed. For example, if one plasma volume is removed, more than one plasma volume may be infused back to the individual receiving the plasmapheresis. Once the plasmapheresis therapy is completed, a second sample is obtained, and a second particle level is determined using the assays described herein. Should a level of particles found in the blood of the individual be determined, after the plasmapheresis therapy, not to have been lowered sufficiently (as compared to a target level or to a reference level), additional plasmapheresis therapy is administered with repeated sampling and measuring of particle level with the plasmapheresis continuing until the level is decreased by a baseline or target level.

In some embodiments, exchange fluid is infused into the individual receiving plasmapheresis simultaneously with the withdrawal of the whole blood, or peripheral blood and removal of the particles so that the exchange fluid reconstitutes some of the plasma volume during the procedure. It is not, therefore, possible to remove the entire plasma volume during a plasmapheresis procedure because, to some degree, plasma is being continuously replenished as it is being removed. As used herein, “plasma volume” refers to the entire volume of plasma within the whole blood of an individual, which can be calculated using numerous methods that are well known for calculating plasma volume. In a plasmapheresis procedure, a volume of plasma that is equal to about 1 plasma volume is removed from the individual receiving plasmapheresis, or 1 plasma volume to 1.5 plasma volumes may be removed in a plasmapheresis procedure. During a plasmapheresis administration as described herein, a volume of exchange fluid is returned to the patient that is about equal to the amount of plasma volume that is withdrawn from the patient so that the removed plasma volume (that is not returned to the patient) is “exchanged” with the exchange fluid that is returned to the patient and replaces the volume of plasma that is removed. While the exchange fluid in some embodiments may be more or less than the plasma volume removed, again, it is about equal in volume. For example, where one plasma volume is removed from an individual in a method described herein, a volume of exchange fluid equal (or about equal) to one plasma volume is returned to the individual.

As described herein, because exchange fluid is simultaneously infused into an individual during a plasmapheresis procedure, the method of removing anywhere from 1-1.5 plasma volumes during a plasmapheresis procedure does not mean that the plasma and its contents are completely removed even though a volume equal to 1-1.5 plasma volumes is removed, because the removed volume includes exchange fluid that had been infused to the individual and then removed as part of the total volume removed. According to Winters (Hematology Am Soc Hematol Educ Program, 2012; 2012(1): 7-12), in a plasmapheresis treatment where 1-1.5 of plasma volume exchanged, approximately 60%-70% of substances present in the plasma at the start of plasmapheresis will be removed which means that approximately 30-40% of substances present in the plasma at the start of plasmapheresis will remain in the body of the individual receiving plasmapheresis following a single plasmapheresis treatment.

Depending on the weight of the individual receiving plasmapheresis, the volume of plasma removed can be between approximately 2 L to 4 L. When 2 L to 4 L of plasma is removed during plasmapheresis, the volume of whole blood or peripheral blood that is withdrawn from the patient is greater than 2.0 L to 4.0 L, which is to say that the withdrawn whole blood or peripheral blood volume contains the volume of plasma to be removed and therefore the whole blood volume withdrawn is larger than the volume of plasma that is removed. It should be understood that plasma volume in an individual is dependent on a number of factors, including weight and gender, and so 2 L to 4 L is used here as a non-limiting example of a range of plasma that might be removed in a plasmapheresis procedure. plasmapheresis, in some instances, can include removal of less than 2 L of plasma or more than 4 L of plasma.

Blood can be withdrawn from a blood vessel of the patient; for example, a peripheral blood vessel (also referred to herein as peripheral blood), a central blood vessel, or a combination thereof. Since the blood flow from the patient into the apheresis device is steady and faster than 50 mL/min, the site of vascular access is a blood vessel capable of withstanding high negative pressure without collapsing.

Moreover, a site of vascular access for receiving an exchange fluid or a mixture that includes an exchange fluid and one or more other components is another blood vessel (or another peripheral or central access point) capable of tolerating relatively high positive pressure. In some embodiments of the methods described herein, whole blood, or peripheral blood, can be withdrawn using a large-bore needle or cannula from a patient's peripheral vein, such as the antecubital fossa, the basilica vein, or the cephalic vein. Additionally, if determined by a plasmapheresis provider to be a vascular access point, whole blood, or peripheral blood, can also be withdrawn by cannulation of a radial artery of an individual receiving plasmapheresis. If determined by a plasmapheresis provider to be a vascular access point, whole blood, or peripheral blood, can be withdrawn using an intravascular or implantable device such as a central venous catheter (CVC), an arteriovenous (AV) shunt, an AV fistulae, or a port-CVC. For example, whole blood, or peripheral blood, can be withdrawn from an internal jugular vein, a subclavian vein, or a femoral vein or artery of the individual receiving plasmapheresis. Blood from an individual receiving plasmapheresis can be withdrawn at a rate of approximately 90 mL/min. However, it is also suitable, in the methods described herein, that blood from an individual receiving plasmapheresis is withdrawn at a rate of between approximately 90 mL/min and 135 mL/min. It should be understood that, under some conditions, withdrawal at a rate of less than 90 mL/min or more than 135 mL/min may be a therapeutic rate for the individual receiving plasmapheresis. As previously discussed, a site of vascular access for receiving a fluid to be returned to an individual receiving plasmapheresis, which may comprise, for example, an exchange fluid, a cellular fraction, a therapeutic, or a blood product and mixtures thereof, is different from a site of vascular access for initial blood withdrawal. For example, a cannula or catheter extending from or otherwise coupled to an apheresis device can be used to deliver a fluid to be returned to an individual receiving plasmapheresis comprising a cellular fraction and an exchange fluid to a blood vessel in an arm, hand, neck, or chest of an individual receiving plasmapheresis.

In an exemplary method for removing particles from an individual, a plasmapheresis method can comprise one or a plurality of plasmapheresis therapies. A plasmapheresis method can begin with the step of identifying an individual in need of a plasmapheresis treatment. The individual in need of a plasmapheresis treatment has an elevated level of particles compared to a control individual or compared to a target or baseline level of particles. The treatment method further includes withdrawing whole blood, or peripheral blood, from a blood vessel of the individual receiving plasmapheresis using an apheresis device or other technique for withdrawing blood. The plasmapheresis method further comprises separating the whole blood, or peripheral blood, withdrawn into a cellular fraction and a nanoparticle fraction using the apheresis device or other technique for separating blood components. The plasmapheresis method can further comprise infusing back to the individual receiving plasmapheresis an exchange fluid and the cellular fraction while removing the particle fraction from the individual. The particle fraction also contains plasma. A volume of exchange fluid returned to the individual may be approximately equal to a volume of plasma that is removed. For example, if one plasma volume is removed from an individual receiving plasmapheresis, in some methods described herein, the volume of an exchange fluid returned to the individual will also be approximately equal to one plasma volume. Alternatively, the volume of exchange fluid returned may exceed the amount of plasma volume removed. For example, if one plasma volume is removed, more than one plasma volume can be infused back to the individual receiving the plasmapheresis. The volume of plasma removed can be from 0.5 to 2 volumes. If a particle level is above 500-800 particles/μL, the frequency of plasmapheresis is increased from two times per week to three or four times per week, from once a month to once a week, or from once every other month to once every month, or other increase in frequency.

Before, during, or after plasmapheresis, a sample can be analyzed to detect particles, guiding treatment decisions. The analysis may reveal different amounts of particles in the sample. In some embodiments, low levels of particles, such as less than 100 particles/μL of blood, may be detected. Moderate levels may range from 100-600 particles/μL, while high levels can exceed 1000 particles/μL. In some embodiments, low levels of particles, such as less than 60, less than 50, less than 40, or less than 30 particles in a blood sample, may be detected. Moderate levels may range from 10-30 particles in a blood sample, while high levels may be greater than 30, greater than 40, greater than 50, or greater than 60 particles in a blood sample. Levels of particles can be measured using particles/μL, particles/mL, particles per sample (e.g., a blood sample), or particles per unit weight (e.g., particles/μg, particles/mg or particles/g). In some embodiments, a pre-treatment level of exogenous particles or the pre-treatment level of particles is between 10 to 150 particles, between 20 to 150 particles, or between 30 to 150 particles. In some embodiments, a pre-treatment level of exogenous particles or the pre-treatment level of particles is greater than 15, 20, 30, 35, 40, 45, or 50 plastic particles. In some embodiments, a pre-treatment level of exogenous particles or the pre-treatment level of particles is greater than 30 plastic particles. In some embodiments, the methods disclosed herein comprise reducing the post-treatment level of exogenous particles or the post-treatment level of particles to a target value. When a level of particles reaches a target level, a healthcare provider can stop further plasmapheresis treatment or may reduce the frequency of treatment to a rate that maintains a target level of particles. In some embodiments, the target level is less than 100, 200, 300, 400, or 500 particles/μL in the circulatory system of the individual. In some embodiments, a target level is between 10-500, 50-100, or 100 and 500 particles/μL in the circulatory system of the individual. A maintenance rate of therapy can be twice a week, once a week, once a month, or once every two, three, four, five, or six months.

The specific target level at which plasmapheresis is no longer needed may vary depending on factors such as the level, type, and size of particles detected, as well as individual patient characteristics. However, in some embodiments, plasmapheresis may not be needed if particle levels fall below 100 particles/μL. In other embodiments, the target level may be set at 0-20, 0-50, 0-100, 0-200, or 200-400 particles/μL. The decision to discontinue plasmapheresis may also consider trends in particle levels over multiple tests rather than relying on a single measurement.

The methods disclosed herein may include repeating the plasmapheresis until the detected levels of particles fall below a target level. The target level may be determined based on various factors, such as the individual's health status, the initial level, the type and size of particles detected, and the potential health risks associated with the detected particles. The target level may be set to a level that is considered safe or acceptable based on current scientific knowledge and health guidelines. In some embodiments, the target level may be adjusted based on new scientific findings or changes in health guidelines.

While there is currently no universally established or accepted level value for particles in human blood, plastic particles in human blood samples have been detected at an average level of 1.6 μg/mL. Levels greater than this can be considered high for plastic particles. The disclosed methods reduce particles in solid organs, including the lung, sputum, liver, spleen, kidney, placenta, gastrointestinal tract, and colon. Particles are also reduced in breast milk, stool, and urine. Levels in these tissues can be, for example, from 82,000 ng/g of PET in infant stool to 0.56 particles/g in the lung. Lung samples can have about 1-3 particles/g of lung tissue. About 30 particles/g can be found in a human colectomy, and levels in a liver, spleen or kidney in some cases can be about 1.4 particles/g. Many of the particles in respiratory organs are fibrous. Plastic particles have also been detected in and on tissues and organs such as blood vessels. The disclosed methods reduce the quantity of inorganic or organic particles in blood, stool, breast milk, sputum, urine, and solid organs such as the lung, liver, spleen, kidney, placenta, and gastrointestinal tract, including the colon.

The methods disclosed herein are also directed to reducing a level of particles adhered to a blood vessel or organ of the individual. The methods reduce the level of particles adhered to blood components such as red blood cells, leukocytes, platelets, and hemoglobin. The methods also reduce the level of particles adhered to a solid organ such as a blood vessel, lung, sputum, liver, spleen, kidney, placenta, and the gastrointestinal tract, including the colon. When an individual is treated according to the described methods, the rate of deposition of particles on a blood vessel or organ of an individual is reduced. The level or amount of particles is also reduced.

As the particles are removed from the whole blood or peripheral blood, the level of particles drops and drives an equilibrium of particles bound to tissue, cells, or organs or located in intracellular or interstitial space compared to particles in the whole blood, or peripheral blood. The drop in the level of particles in the blood drives particles in other areas to move to the bloodstream, and they can then be removed by further plasmapheresis treatments. The amount or level of particles in a tissue, cell, or organ can be measured using a biopsy or imaging technique and then can be compared to an amount or level of particles in whole blood, or peripheral blood, or in a target or reference sample. After treatment, the amount or level of particles is again measured. Even when an individual is not exposed to above-average levels of contamination, the amount or level of particles in the blood can increase, remain the same, or decrease more slowly compared to a reference individual, as particles in non-vascular space, such as those adhering to tissues, organs, or blood vessels, move to vascular space. After multiple treatments, the amount or level of tissue-bound particles is decreased, and the amount or level of tissue-bound particles in the blood is also decreased.

For example, the method can include measuring an initial level of particles adhered to a blood vessel or organ of an individual, performing the disclosed method, measuring a second level of particles adhered to a blood vessel or organ of an individual, and repeating the steps until the second level of particles adhered to a blood vessel or organ of an individual is lower than a baseline or reference value.

The disclosed methods can remove particles at a rate of between 1-99.9% per treatment, per day, per week, or per month. In some embodiments, the methods reduce particle levels by 1-1000 particles/μL per treatment, per day, per week, or per month. The described plasmapheresis methods reduce particles by about 1-3000 particles/μL per treatment, about 100-200, about 200-300, about 300-400, about 400-500, about 500-600, about 600-700, about 700-800, about 800-900, about 900-1000, about 1000-1500, or about 1500-2000 particles/μL per treatment per day, per week, or per month.

Plasmapheresis reduces the level of particles in the blood of an individual by 1% to 99.9%. The level of particles can be reduced from 1%-10%, 10%-20%, 20%-30%, 30%-40%, 40%-50%, 50%-60%, 60%-70%, 70%-80%, 80%-90%, or 90%-99.9%. As plasmapheresis is administered multiple times and the level of particles decreases, a target value of percent reduction is reached. The target value can be 1% to 99.9% reduction. The target value can be 1%-10%, 10%-20%, 20%-30%, 30%-40%, 40%-50%, 50%-60%, 60%-70%, 70%-80%, 80%-90%, or 90%-99.9% reduction in the level of particles compared to an initial level of particles.

In some embodiments, the plasmapheresis is administered once, twice, three times, or four times a week for one, two, three, four, five, six months or longer. In some embodiments, the plasmapheresis is administered once a month, once every other month, once every three months, once every four months, once every five months, or once every six months for three months, four months, five months, six months or longer. In some embodiments, the plasmapheresis is administered twice a week for three months, or once every month for six months. In some embodiments, the plasmapheresis is administered twice a week for three months, or once every month for six months. When at least two administrations are performed per month, in some embodiments at least two administrations are performed in a single week. When the target level is reached, the administration frequency is reduced, for example, from twice a week to once a month, or from once a month to once every other month. Other frequencies of administration are also possible, and these frequencies of administration are not meant to be limiting in any way.

The frequency of the plasmapheresis may be determined based on various factors, such as the individual's health status, the amount and type of particles detected, and the individual's response to the plasmapheresis. In some embodiments, the frequency of the plasmapheresis may be adjusted based on the individual's response to the treatment and the detected level of particles. Different types of particles may adhere to organ tissues, cell membranes, or can be located in intracellular or extracellular spaces and may take time to elute into the bloodstream. Depending on the types, sizes, or level of plastic, the frequency of plasmapheresis is increased or decreased.

Depending on the desired frequency of treatment, the plasmapheresis can be performed for a duration of time, such as at least 1, 2, 3, 4, 5, or 6 months or longer. The duration of the plasmapheresis may be determined based on various factors, such as the individual's health status, the amount and type of particles detected, and the individual's response to the plasmapheresis. In some embodiments, the duration of the plasmapheresis may be adjusted based on the individual's response to the treatment and the detected level of particles.

The methods disclosed herein can include repeating the plasmapheresis until the amount, size, or type of particles detected is lower than or equal to a target or baseline range. The target or baseline range may be determined based on various factors, such as the individual's health status, the type and size of particles found in the population, and the potential health risks associated with the detected particles. The target or baseline range may be set to a level that is considered safe or acceptable based on current scientific knowledge and health guidelines. In some embodiments, the target or baseline range may be adjusted based on new scientific findings or changes in health guidelines.

A target level of particles can vary depending on the occupation, geographical location, target tissue or organ, or initial level of particles in an individual. A target level of particles can be from about 1 to 500 particles/μL, about 50 to 500 particles/μL, about 100 to 500 particles/μL, about 300-500 particles/μL, about 1 to 50 particles/μL, about 50 to 100 particles/μL, about 100 to 200 particles/μL, about 200 to 300 particles/μL, about 300 to 400 particles/μL, or about 400 to 500 particles/μL. In some embodiments, a target level of particles (e.g., exogenous particles) may be less than 60, less than 50, less than 40, or less than 30 particles in a blood sample. In some embodiments, a target level of particles (e.g., exogenous particles) may be from 0-30 particles in a blood sample. Levels of particles can be measured using particles/μL, particles/mL, particles per sample (e.g., a blood sample), or particles per unit weight (e.g., particles/μg, particles/mg or particles/g). In some embodiments, the target level is less than 100, 200, 300, 400, or 500 particles/μL in the circulatory system of the individual. In some embodiments, a target level is between 10-500, 50-100, or 100 and 500 particles/μL in the circulatory system of the individual. A target level can be a reference level taken from an age-matched individual, a comparable individual, or an average level of a comparative population of individuals. A comparative population of individuals is a group of individuals sharing traits such as age, weight, disease burden, lifestyle, occupation, or other traits. A baseline level refers to a level taken before treatment with the described method, as compared to a level taken during or after treatment with the described method. A level of particles described herein can be a level of bead-shaped particles, a level of fiber-shaped particles, a level of sphere-shaped particles, a level of fragment-shaped particles, or a combination thereof. In some embodiments, the target level is from about 1% to 99.9% of a pretreatment level of the particles. A target level can be from about 1% to 20%, 20% to 40%, 40% to 60%, 60% to 80%, or 80% to 99.9% of a baseline level of the particles. The initial level can be higher than the reference level, in which case the treatment is performed to bring the level of particles within 1 to 20%, 1 to 10%, or 1 to 5% of the reference level.

The target or baseline range may be based on any medically desired level of particles determined to decrease health complications resulting from particle exposure. These controls can provide a benchmark for assessing the amount, size, or type of particles associated with a reduced risk of health complications resulting from exposure. By comparing the detected levels in the individual to those controls, the methods can provide useful insights into the individual's exposure to particles relative to their peers. In some embodiments, the amount, size, or type of particles detected in the individual is higher than the target or baseline range. This can indicate that the individual has a higher exposure to particles compared to the controls, which can pose health risks. In such embodiments, the methods disclosed herein include performing plasmapheresis to remove the particles from the individual's body.

A comparison of detected particle levels to a target level, baseline level, or reference database contributes to the treatment of the individual's exposure to particles. A reference database can contain average levels, sizes, and characteristics of particles in a population and can guide the particle removal process. For example, if the detected particle levels are significantly higher than the average levels in a reference database, than a target level, or than a baseline level, the plasmapheresis process may be adjusted to more aggressively remove particles from the individual. Conversely, if the detected particle levels are within the range, the target level, or the baseline level of the reference database, the plasmapheresis process may be adjusted to maintain the particle levels within a safe range. For example, if the particle level is high, a greater volume of plasma is exchanged or the frequency of plasmapheresis is increased. In one aspect, if a particle level is above 500-800 particles/μL, 1.5 to 2 volumes of plasma are exchanged instead of 1 volume of plasma being exchanged. In another aspect, the plasmapheresis is performed more frequently, such as four to eight times per month instead of two times per month. These values are merely descriptive, and other values are meant to be included, such as 0.5 volumes, 1 volume, 1.5 volumes, or 2 volumes of plasma, and 1, 2, 3, 4, 5, 6, 7, 8, 9, 10, 11, or 12 times per month or more.

The decision to discontinue plasmapheresis can be based on trends in levels over multiple tests rather than relying on a single measurement. This approach recognizes that levels of particles in an individual's blood may fluctuate over time due to various factors such as recent exposure, diet, or other environmental influences. By considering trends across multiple tests, healthcare providers can better assess the overall effectiveness of the plasmapheresis treatment and make more informed decisions about continuing or discontinuing therapy.

For example, if an individual's levels show a consistent downward trend over several tests, even if they have not yet reached the predetermined target level, the decrease in level can indicate that the treatment is effective and can be continued. Conversely, if levels remain consistently high or show only a slight decrease despite multiple plasmapheresis sessions, it may suggest that the treatment approach can be reevaluated or that additional interventions may be added.

This trend-based approach also helps account for potential measurement variabilities or temporary fluctuations that could occur in a single test. It provides a more robust and reliable assessment of the individual's response to treatment and their overall particle burden. Additionally, monitoring trends over time can help identify patterns in accumulation or clearance that may be distinct in each individual, allowing for more personalized treatment strategies.

Measuring particle levels over time can reveal individual-specific patterns of accumulation and clearance that may not be apparent from single measurements. These patterns can be influenced by factors such as an individual's lifestyle, diet, occupation, or distinct physiological characteristics. For example, some individuals can show rapid clearance of different types of particles after plasmapheresis, while others can exhibit slower clearance rates or faster re-accumulation.

By identifying these individual-specific patterns, healthcare providers can tailor the frequency and intensity of plasmapheresis treatments to each patient's individual symptoms. For instance, a patient showing rapid re-accumulation of particles may require more frequent plasmapheresis sessions, while a patient with slower accumulation rates might benefit from less frequent treatments. Additionally, understanding individual clearance patterns can help in determining the timing for follow-up measurements and adjusting treatment protocols accordingly.

Furthermore, monitoring particle levels over time can provide insights into the effectiveness of complementary interventions aimed at reducing particle exposure or enhancing natural clearance mechanisms. Complementary interventions or treatments could include dietary modifications, lifestyle changes, or the use of supplements or medications that may influence particle accumulation or elimination. This personalized approach based on long-term monitoring leads to more efficient and effective treatment strategies, improving patient outcomes while optimizing resource utilization in healthcare settings.

As research in this field progresses, any target level can be regularly reviewed and updated based on new scientific evidence and potential health impacts of particles. The determination of a safe or acceptable level may also consider factors such as the type of material, particle size, and potential long-term effects of accumulation in the body.

The methods disclosed herein can effectively reduce the amount of particles in an individual's body. For instance, after performing plasmapheresis, the amount of particles detected in the individual's body can be reduced by 1% to 99.9% compared to a baseline. In some embodiments, the amount of particles can be reduced by 90%, 80%, 70%, 60%, 50%, 40%, 30%, 20%, 10%, or 1% compared to the pretreatment level. This reduction can be quantified by comparing the amount of particles detected before and after the plasmapheresis procedure. In some embodiments, the methods can be repeated until the amount of particles detected falls below a target level, indicating a significant reduction in the individual's exposure to particles.

### Exchange Fluids

An exchange fluid may comprise any fluid that is suitable for use in intravenous fluid administration. For example, non-limiting examples of fluids suitable for intravascular administration with the administration of plasmapheresis as described herein are commonly referred to as isotonic fluids and include normal saline (i.e., a 0.9% saline solution) and lactated Ringer's. An exchange fluid solution suitable for use in plasmapheresis as described herein may further include albumin such as human albumin. For example, an exchange fluid may comprise a normal saline solution that includes 5% albumin by weight. In some embodiments, the exchange fluid used in the plasmapheresis process may comprise albumin. Albumin is a protein that is naturally found in blood plasma and serves various functions in the body, such as maintaining osmotic pressure and transporting various substances. In some embodiments, the albumin in the exchange fluid may be human serum albumin. The concentration of albumin in the exchange fluid may be about 1% to about 8%, about 1%, about 2%, about 3%, about 4%, about 5%, about 6%, about 7%, about 8%, about 9%, or about 10%. In some embodiments, the albumin may be administered at a dose of about 0.5 g/kg to about 2 g/kg of bodyweight of the individual.

A source of albumin is human derived albumin also referred to as human serum albumin (HSA). As an example, an exchange fluid suitable for use with plasmapheresis may comprise a sterile liquid preparation comprising a concentration of human-derived protein in the amount of 50 g per 1000 ml of the sterile liquid preparation wherein at least 96% of the human-derived protein is human serum albumin protein. Besides the human-derived serum albumin protein, the remainder of the HSA preparation can comprise a saline solution and small amounts of potassium, N-acetyl-DL-tryptophan, caprylic acid, or a combination thereof. A 5% HSA preparation of an exchange fluid can be an FDA-approved 5% HSA preparation. More specifically, the 5% HSA preparation can be manufactured by an FDA-approved procedure such as the Cohn-Oncley cold ethanol fractionation procedure followed by ultra-filtration and pasteurization. Replacing plasma withdrawn from an individual receiving plasmapheresis with 5% HSA is useful for regulating and stabilizing the volume of circulatory blood within the individual.

An exchange fluid may be mixed or combined in any suitable way with the cellular fraction of the whole blood that is withdrawn during plasmapheresis, which remains after separation of plasma and which is returned to the individual receiving plasmapheresis during the procedure. An exchange fluid suitable for use with plasmapheresis may also include one or more therapeutics. For example, a therapeutic that reduces inflammation may be provided with plasmapheresis by, for example, mixing the therapeutic with an exchange fluid. An exchange fluid for plasmapheresis may also comprise or be mixed together with one or more blood products (i.e., not including the cellular fraction that is returned) including but not limited to fresh frozen plasma and platelets. It should be understood, that when mixed with an exchange fluid, therapeutics and blood products will, at least to some degree, be withdrawn back from the individual receiving the plasmapheresis and as such it may be beneficial to administer or infuse a therapeutic and/or blood product after completing the blood withdrawal from the individual so that the therapeutic and/or blood product will not be withdrawn from the individual during the plasmapheresis therapy.

As stated, in a plasmapheresis procedure, a volume of whole blood, or peripheral blood, is withdrawn from an individual and a portion of it is returned to the individual along with an exchange fluid. The exchange fluid is returned to the individual simultaneously with the withdrawal of the whole blood which makes calculations related to the plasmapheresis process not simple and straightforward.

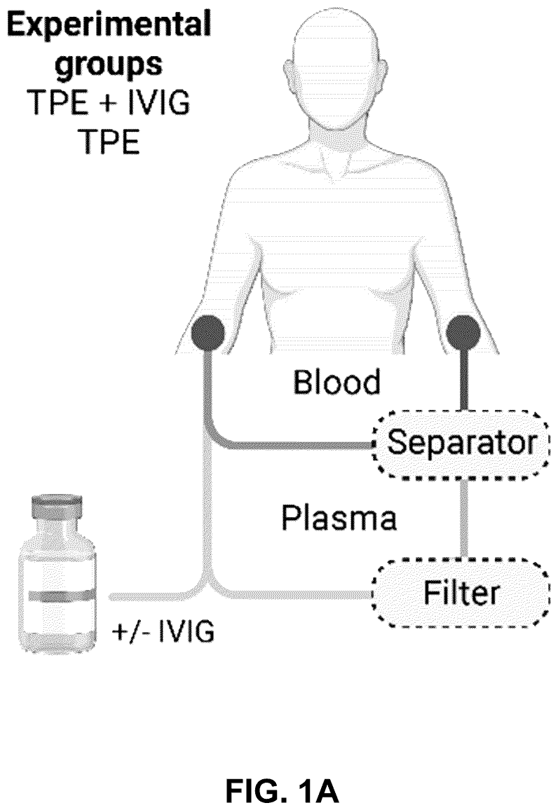

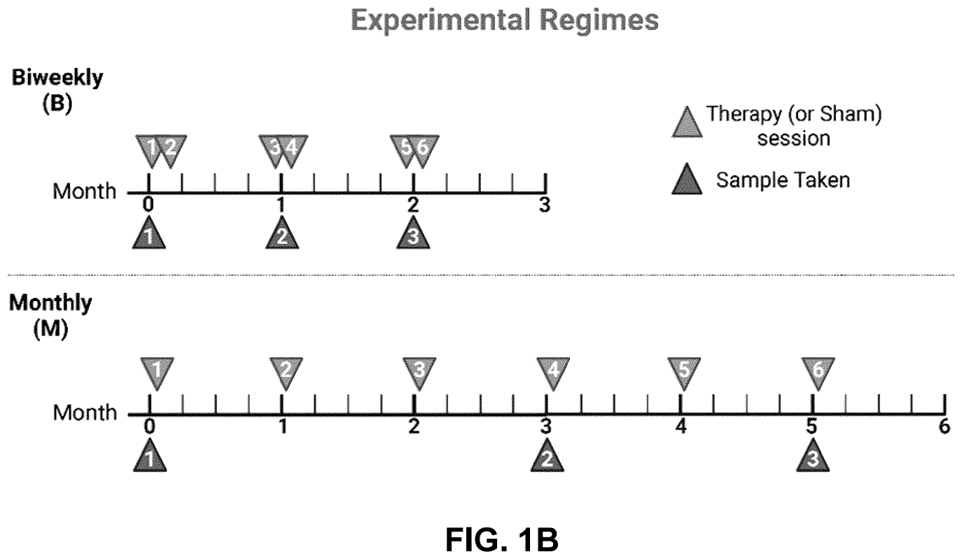

The exchange fluid used in the plasmapheresis process may comprise immunoglobulins. Immunoglobulins, also known as antibodies, are proteins produced by the immune system to neutralize pathogens such as bacteria and viruses. In some embodiments, the immunoglobulins in the exchange fluid can be intravenous immunoglobulin (IVIG), which is a product made up of antibodies that can be given intravenously (through a vein). IVIG is used to treat various autoimmune, infectious, and idiopathic diseases. In some embodiments, the IVIG can be administered at a dose of about 0.5 g/kg to about 2 g/kg of bodyweight of the individual. The IVIG can be administered at a dose of about 0.5 g/kg, 1 g/kg, 1.5 g/kg, or 2 g/kg. In some embodiments, the IVIG can be administered at a dose of about 1 g, about 2 g, about 3 g, about 4 g, or about 5 g per individual. In some embodiments, the IVIG is administered at 2 g per individual. In some embodiments, the IVIG is administered in the exchange fluid during plasmapheresis. In some embodiments, the IVIG is administered concurrently with the exchange fluid during plasmapheresis. In some embodiments, IVIG is not administered with the plasmapheresis. In some embodiments, the IVIG is administered after the plasmapheresis (e.g., within 24 hours, within 48 hours, or within 72 hours of the plasmapheresis). FIG. 1A and FIG. 1B is a schematic diagram showing TPE treatment and regimes.

A plasmapheresis treatment as described herein can further comprise delivering a therapeutically-effective dosage of intravenous immunoglobulin (IVIG) to a blood vessel of the individual receiving the plasmapheresis treatment. In certain plasmapheresis treatments as described herein, IVIG is delivered to an individual receiving plasmapheresis after returning the cellular fraction and the exchange fluid to the individual receiving the plasmapheresis. This is to say that a therapeutically-effective dosage of IVIG may be provided to an individual separately from infusion of the cellular fraction and the exchange fluid to the individual receiving the plasmapheresis. For example, IVIG may be infused in the individual receiving plasmapheresis after a plasmapheresis treatment is completed so that any infused IVIG will not be removed in the plasmapheresis process. It is also possible to administer IVIG concurrently with the administration of plasmapheresis.

In some embodiments, a therapeutically-effective dosage of IVIG may be approximately 2.0 g of IVIG per individual receiving plasmapheresis. The IVIG delivered can contain certain antibodies and cytokines which can have a positive effect on the immune system of the individual receiving plasmapheresis and may contribute to establishing an optimal systemic environment for cell growth. As stated above, it is suitable for administration of a therapeutic such as IVIG mixed together with an exchange fluid and/or a cellular fraction during a portion of the plasmapheresis procedure when blood is being withdrawn or it is also suitable to administer IVIG to the individual as part of a method for administering plasmapheresis as described herein when blood is no longer being withdrawn from the individual receiving plasmapheresis. The therapeutically-effective dosage of IVIG can be any FDA-approved IVIG or immune globulin intravenous (IGIV) infusion or preparation. For example, the IVIG can comprise primarily of gamma globulins.

The steps described do not require the particular order shown to achieve the desired result. Moreover, certain steps or processes may be omitted or occur in parallel in order to achieve the desired result.

Each of the plasmapheresis treatment sessions can comprise the steps of withdrawing whole blood from a blood vessel of a patient using an apheresis device, separating the whole blood withdrawn into a cellular fraction and a plasma fraction using the apheresis device, admixing the cellular fraction with an exchange fluid comprising albumin derived from human plasma, returning the mixture comprising the cellular fraction and the exchange fluid to the blood vessel of the patient using an apheresis device, and delivering a therapeutically-effective dosage of intravenous immunoglobulin (IVIG) to the blood vessel of the individual receiving plasmapheresis after returning the mixture comprising the cellular fraction and the exchange fluid to a blood vessel of the individual.

Micro- and nanoparticles are associated with health risks which can be reduced both by removal of the particles and further by administration of anti-immunogenic compounds. Micro-and nanoparticles can induce erythrocyte hemolysis, cytotoxicity and genotoxicity in the circulatory system, as well as inducing thrombus formation. These small particles contribute to oxidative stress and neuroinflammation. The effects of these particles are seen throughout various organ systems of the human body, including the reproductive system, locomotor, digestive, respiratory, and endocrine systems, in addition to the nervous system and circulatory system.

As used herein, the term “circulatory system” refers to both the cardiovascular system and the lymphatic system. The cardiovascular system refers to the network of the heart, blood vessels, and blood that circulates oxygen, nutrients, hormones, and waste products throughout the body to maintain homeostasis and support cellular function. The lymphatic system transports white blood cells and lymph from tissues to the veins, helping to maintain fluid balance in the body. Plasmapheresis can affect the circulatory system to remove micro- and nanoparticles.

Removal of micro- and nanoparticles treats inflammatory effects, cytotoxic effects and oxidative stress caused by the particles and by toxic substances adsorbed on the plastic. The addition of immunoglobulins to the exchange fluid can further treat these symptoms and health risks.

The exchange fluid may comprise a combination of albumin and immunoglobulins. This combination provides the benefits of both albumin and immunoglobulins, such as maintaining osmotic pressure, transporting various substances, and enhancing the immune response.

An anti-inflammatory or immune-modulating therapeutic can be used synergistically with plasmapheresis. This therapeutic can be administered before, during, or after the plasmapheresis process. The anti-inflammatory or immune-modulating therapeutic reduces inflammation and modulates the immune response, which enhances the effectiveness of the plasmapheresis process in removing particles from the individual's body. Various types of anti-inflammatory or immune-modulating therapeutics can be used, including but not limited to nonsteroidal anti-inflammatory drugs (NSAIDs), corticosteroids, immunosuppressants, and biologics.

A particle-binding agent can be added to the plasma, including chitosan, activated carbon, biochar, a silica nanoparticle, a magnetic nanoparticle, or a combination thereof. A coating for adsorbing particles can also be used during plasmapheresis. For example, a coating such as polystyrene sulfonate or polyaspartate can be added to adsorb plastic particles. When plasmapheresis is performed using a membrane, and particles are removed using a semipermeable membrane, a binding agent can be added to the cellular fraction or to the particle fraction before, during, or after plasmapheresis. A particle-specific adsorption column can also be used.

### Particle Removal Systems

Systems for detecting and removing particles from an individual may include a plasmapheresis device, a measuring device, and a processor. The plasmapheresis device may be configured to separate plasma from whole blood or peripheral blood. The measuring device may be configured to analyze samples, including particles. The processor may be configured to quantify particles. In some embodiments, the measuring device is a flow cytometer, and the samples are labeled with a fluorescent dye that adsorbs or binds to the particle. The dye can be any of the dyes described herein, including Nile Red. The processor would then be configured to quantify particles based on fluorescence data from the flow cytometer.

The system may further include a display configured to show real-time quantification of particles during the plasmapheresis. This feature may allow healthcare providers or the individual to monitor the progress of the particle removal process in real-time, providing immediate feedback on the effectiveness of the plasmapheresis.

The system may include a storage unit for preserving plasma samples for later analysis. This feature may allow for a more detailed and comprehensive analysis of the particles, which may not be feasible to perform in real-time during the plasmapheresis. The stored particle fraction can be analyzed at a later time using various techniques known in the art, such as immunoassays, flow cytometry, mass spectrometry, Raman spectroscopy, SPR, antibody-based detection methods, or FTIR.

The processor of the system may be further configured to compare detected particle levels to a reference database. The reference database may include information on particle levels in a population of individuals, which can be used as a benchmark for assessing the particle levels in the individual. The comparison of detected particle levels to the reference database may provide useful insights into the individual's exposure to particles relative to the general population, and may help guide the particle removal process. For example, if the detected particle levels are significantly higher than the average levels in the reference database, the plasmapheresis process may be adjusted to more aggressively remove particles from the individual. Conversely, if the detected particle levels are within the range of the reference database, the plasmapheresis process may be adjusted to maintain the particle levels within a safe range. For example, if the particle level is high, a greater volume of plasma is exchanged or frequency of plasmapheresis is increased. In one aspect, if a particle level is above 500-800 particles/μL, 1.5 to 2 volumes of plasma are exchanged instead of 1 volume of plasma being exchanged. In another aspect, if a particle level is above 500-800 particles/μL, the frequency of plasmapheresis is increased from two times per week to three or four times per week, or from once a month to once a week, or from once every other month to once every month.

### Modeling Blood Dilution in Plasmapheresis

Also described herein is a method for removing particles by carrying out a blood dilution achieved by a plasmapheresis treatment (e.g., therapeutic plasma exchange (TPE)). As described herein, a particle fraction can be removed and exchange fluid added. The removal of a particle fraction (even with partial replacement) together with the addition of exchange fluid lowers the levels of one or more constituents of plasma in an individual receiving plasmapheresis treatment. In summary, solute (the plasma content) is decreased while the solvent (the liquid portion of plasma replaced by the exchange fluid) remains the same. In this way, in addition to removing a particle fraction, the methods described herein dilute the particles that remain in the blood of the individual receiving plasmapheresis following a plasmapheresis treatment.

In some embodiments, a plasmapheresis method can begin with the step of identifying an individual in need of a plasmapheresis therapy. A blood sample may be taken from the individual, either before initiation of the plasmapheresis, or from the whole blood that is withdrawn and used to determine a pre-treatment level of exogenous particles (e.g., a pre-treatment level of plastic particles or particles) (e.g., by measuring an exogenous particle level). The plasmapheresis method can further comprise withdrawing whole blood from a blood vessel of the individual receiving plasmapheresis using an apheresis device or other technique for withdrawing blood. The plasmapheresis method can further comprise separating the whole blood withdrawn into a cellular fraction and a plasma fraction using the apheresis device or other technique for separating blood components. The treatment method can further comprise infusing back to the individual receiving plasmapheresis an exchange fluid and the cellular fraction while removing the plasma fraction from the individual. An amount of exchange fluid returned to the individual may be approximately equal to an amount of plasma that is removed. For example, if one plasma volume is removed from an individual receiving plasmapheresis, in certain methods described herein, an amount of an exchange fluid returned to the individual will also be approximately equal to one plasma volume. Alternatively, an amount of exchange fluid returned may also exceed the amount of plasma volume removed. For example, if one plasma volume is removed, more than one plasma volume may be infused back to the individual receiving the plasmapheresis. Once the plasmapheresis therapy is completed, a second sample is obtained and a post-treatment level of exogenous particles (e.g., a post-treatment level of plastic particles or particles) is established (e.g., is measured). Should the post-treatment level of exogenous particles (e.g., a post-treatment level of plastic particles or particles) in the blood of the individual be determined after the plasmapheresis therapy not to have been lowered sufficiently (as compared to the pre-treatment level of exogenous particles (e.g., a pre-treatment level of plastic particles or particles)), additional plasmapheresis therapy may be administered with repeated sampling and measuring of pre-treatment level of exogenous particles (e.g., a pre-treatment level of plastic particles or particles) and continuing until the post-treatment level of exogenous particles (e.g., a post-treatment level of plastic particles or particles) is decreased by an amount that is sought. In some embodiments, the post-treatment level of exogenous particles (e.g., a post-treatment level of plastic particles or particles) is decreased by from about 10% to about 30%, about 20% to about 40%, about 30% to about 50%, about 40% to about 60%, about 50% to about 70%, or about 40% to about 80% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by from about 30% to about 60% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by up to 10%, 15%, 20%, 25%, 30%, 35%, 40%, 45%, 50%, 55%, 60%, 65%, 70%, 75%, 80%, 85%, or 90% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by at least 10%, 15%, 20%, 25%, 30%, 35%, 40%, 45%, 50%, 55%, 60%, 65%, 70%, 75%, 80%, 85%, or 90% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by at least 10% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by at least 20% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by at least 30% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by at least 40% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by at least 50% as compared to the pre-treatment level of exogenous particles (e.g., a pre-treatment level of plastic particles or particles). In some embodiments, the post-treatment level of exogenous particles (e.g., a post-treatment level of plastic particles or particles) is decreased by at least 60% as compared to the pre-treatment level of exogenous particles (e.g., a pre-treatment level of plastic particles or particles).

Described herein are methods of reducing a level of exogenous particles in an individual by plasmapheresis, the method comprising: (a) withdrawing a volume of whole blood from the individual having a pre-treatment level of exogenous particles; (b) separating the volume of whole blood into a cellular fraction and an exogenous particle fraction, wherein the exogenous particle fraction comprises a level of exogenous particles; and (c) returning the cellular fraction and an exchange fluid to a circulatory system of the individual, thereby reducing the pre-treatment level of exogenous particles to a post-treatment level of exogenous particles in the individual. In some embodiments, the method may comprise administering plasmapheresis comprising performing steps (a) through (c). In some embodiments, the method comprises repeating steps (a) through (c) until the quantitative difference between the post-treatment level of exogenous particles in the individual and the pre-treatment level of exogenous particles in the individual is a target value. In some embodiments, the method comprises repeating steps (a) through (c) until the post-treatment level of exogenous particles or the post-treatment level of particles is below a threshold level in one or more of the following: the circulatory system of the individual; the organ of the individual; or the blood vessel wall of the individual.

Described herein are methods of treating a condition associated with exposure to exogenous particles in an individual in need thereof, the method comprising: (a) measuring a pre-treatment level of the exogenous particles in a blood sample of the individual prior to performing plasmapheresis; (b) withdrawing a volume of whole blood from the individual; (c) separating the volume of whole blood into a cellular fraction and an exogenous particle fraction, wherein the exogenous particle fraction comprises a level of exogenous particles; and (d) returning the cellular fraction and an exchange fluid to a circulatory system of the individual, thereby reducing the pre-treatment level of exogenous particles to a post-treatment level of exogenous particles in the individual. In some embodiments, the method may comprise administering plasmapheresis comprising performing steps (b) through (d). In some embodiments, the method comprises repeating steps (b) through (d) until the quantitative difference between the post-treatment level of exogenous particles in the individual and the pre-treatment level of exogenous particles in the individual is a target value. In some embodiments, the method comprises repeating steps (b) through (d) until the post-treatment level of exogenous particles or the post-treatment level of particles is below a threshold level in one or more of the following: the circulatory system of the individual; the organ of the individual; or the blood vessel wall of the individual.

Described herein are methods method of providing plasmapheresis to an individual for reducing a level of exogenous particles in the individual, comprising the steps of: (a) measuring, before an administration of plasmapheresis, a level of exogenous particles in a blood sample of the individual, thereby generating a pre-treatment level of exogenous particles in the individual; (b) withdrawing a volume of whole blood from the individual; (c) separating the volume of whole blood into a cellular fraction and an exogenous particle fraction, wherein the exogenous particle fraction comprises a level of exogenous particles; and (d) returning the cellular fraction and an exchange fluid to a circulatory system of the individual, (e) measuring, following step (d), a level of particles in a blood sample of the individual, thereby generating a post-treatment level of exogenous particles in the individual; thereby reducing the pre-treatment level of exogenous particles to the post-treatment level of exogenous particles in the individual. In some embodiments, the method may comprise administering plasmapheresis comprising performing steps (b) through (d). In some embodiments, the method comprises repeating steps (b) through (d) until the quantitative difference between the post-treatment level of exogenous particles in the individual and the pre-treatment level of exogenous particles in the individual is a target value. In some embodiments, the method further comprises determining a quantitative decrease of the post-treatment level of exogenous particles in the individual compared with the pre-treatment level of exogenous particles in the individual. In some embodiments, the method comprises repeating steps (b) through (d) until the post-treatment level of exogenous particles or the post-treatment level of particles is below a threshold level in one or more of the following: the circulatory system of the individual; the organ of the individual; or the blood vessel wall of the individual.

Described herein are methods of reducing a level of particles in an individual by plasmapheresis, the method comprising: (a) withdrawing a volume of whole blood from the individual with a pre-treatment level of particles; (b) separating the volume of whole blood into a cellular fraction and a particle fraction, wherein the particle fraction comprises a level of particles; and (c) returning the cellular fraction and an exchange fluid to a circulatory system of the individual, thereby reducing the level of particles in the individual to a post-treatment level of particles. In some embodiments, the method may comprise administering plasmapheresis comprising performing steps (a) through (c). In some embodiments, the particles are exogenous particles. In some embodiments, the method comprises repeating steps (a) through (c) until the quantitative difference between the post-treatment level of exogenous particles in the individual and the pre-treatment level of exogenous particles in the individual is a target value. In some embodiments, the method comprises repeating steps (a) through (c) until the post-treatment level of exogenous particles or the post-treatment level of particles is below a threshold level in one or more of the following: the circulatory system of the individual; the organ of the individual; or the blood vessel wall of the individual.

Described herein are methods reducing a level of exogenous particles in an individual by plasmapheresis, the method comprising: (a) withdrawing a volume of whole blood from the individual with a pre-treatment level of exogenous particles; (b) separating the volume of whole blood into a cellular fraction and an exogenous particle fraction, wherein the exogenous particle fraction comprises a level of exogenous particles; and (c) returning the cellular fraction and an exchange fluid to a circulatory system of the individual, thereby reducing the level of exogenous particles in the individual to a post-treatment level of exogenous particles. In some embodiments, the method may comprise administering plasmapheresis comprising performing steps (a) through (c). In some embodiments, the method comprises repeating steps (a) through (c) until the quantitative difference between the post-treatment level of exogenous particles in the individual and the pre-treatment level of exogenous particles in the individual is a target value. In some embodiments, the method comprises repeating steps (a) through (c) until the post-treatment level of exogenous particles or the post-treatment level of particles is below a threshold level in one or more of the following: the circulatory system of the individual; the organ of the individual; or the blood vessel wall of the individual.

Described herein are methods of reducing a level of exogenous particles in an individual by plasmapheresis, the method comprising: (a) measuring a pre-treatment level of exogenous particles in a blood sample of the individual prior to performing plasmapheresis; (b) withdrawing a volume of whole blood from the individual; (c) separating the volume of whole blood into a cellular fraction and an exogenous particle fraction, wherein the exogenous particle fraction comprises a level of exogenous particles; and (d) returning the cellular fraction and an exchange fluid to a circulatory system of the individual, thereby reducing the pre-treatment level of exogenous particles to a post-treatment level of exogenous particles in the individual. In some embodiments, the method may comprise administering plasmapheresis comprising performing steps (b) through (d). In some embodiments, the method comprises repeating steps (b) through (d) until the quantitative difference between the post-treatment level of exogenous particles in the individual and the pre-treatment level of exogenous particles in the individual is a target value. In some embodiments, the method comprises repeating steps (b) through (d) until the post-treatment level of exogenous particles or the post-treatment level of particles is below a threshold level in one or more of the following: the circulatory system of the individual; the organ of the individual; or the blood vessel wall of the individual.

In some embodiments, the pre-treatment level of exogenous particles or the pre-treatment level of particles is between 10 to 150 particles, between 20 to 150 particles, or between 30 to 150 particles. In some embodiments, the pre-treatment level of exogenous particles or the pre-treatment level of particles is greater than 15, 20, 30, 35, 40, 45, or 50 plastic particles. In some embodiments, the pre-treatment level of exogenous particles or the pre-treatment level of particles is greater than 30 plastic particles.

In some embodiments, the method further comprises measuring any one of the pre-treatment level of exogenous particles, the pre-treatment level of particles, the post-treatment level of exogenous particles, or the post-treatment level of particles in the circulatory system of the individual. In some embodiments, measuring any one of the pre-treatment level of exogenous particles, the pre-treatment level of particles, the post-treatment level of exogenous particles, or the post-treatment level of particles in an organ of the individual. In some embodiments, measuring any one of the pre-treatment level of exogenous particles, the pre-treatment level of particles, the post-treatment level of exogenous particles, or the post-treatment level of particles in a blood vessel of the individual.

In some embodiments, reducing the post-treatment level of exogenous particles or the post-treatment level of particles to a target value. In some embodiments, the target level is less than 100, 200, 300, 400, or 500 particles/μL in the circulatory system of the individual. In some embodiments, target level is between 10-500, 50-100, or 100 and 500 particles/μL in the circulatory system of the individual. In some embodiments, the post-treatment level of exogenous particles or the post-treatment level of particles is decreased by from about 10% to about 30%, about 20% to about 40%, about 30% to about 50%, about 40% to about 60%, about 50% to about 70%, or about 40% to about 80% as compared to the pre-treatment level of exogenous particles or the pre-treatment level of particles. In some embodiments, the post-treatment level of exogenous particles or the post-treatment level of particles is decreased by from about 30% to about 60% as compared to the pre-treatment level of exogenous particles or the pre-treatment level of particles. In some embodiments, the post-treatment level of exogenous particles or the post-treatment level of particles is decreased by up to 10%, 15%, 20%, 25%, 30%, 35%, 40%, 45%, 50%, 55%, 60%, 65%, 70%, 75%, 80%, 85%, or 90% as compared to the pre-treatment level of exogenous particles or the pre-treatment level of particles. In some embodiments, the post-treatment level of exogenous particles or the post-treatment level of particles is decreased by at least 10%, 15%, 20%, 25%, 30%, 35%, 40%, 45%, 50%, 55%, 60%, 65%, 70%, 75%, 80%, 85%, or 90% as compared to the pre-treatment level of exogenous particles or the pre-treatment level of particles. In some embodiments, the post-treatment level of exogenous particles or the post-treatment level of particles is decreased by at least 40% as compared to the pre-treatment level of exogenous particles or the pre-treatment level of particles.

In some embodiments, the administering of the plasmapheresis comprises exchanging at least one unit of plasma volume. In some embodiments, administering the plasmapheresis over a plurality of treatment sessions. In some embodiments, administering the plasmapheresis over a single treatment session.

In some embodiments, the method further comprises administering intravenous immunoglobulin to the individual. In some embodiments, the intravenous immunoglobulin is administered at a dose of 1 gram, 2 grams, 3 grams, 4 grams, or 5 grams per individual. In some embodiments, the intravenous immunoglobulin is administered at a dose of 2 grams per individual. In some embodiments, the method comprises administering intravenous immunoglobulin to the individual following plasmapheresis. For example, the intravenous immunoglobulin may be administered to the individual directly (e.g., immediately) following plasmapheresis. In another example, the intravenous immunoglobulin may be administered to the individual within 24 hours, within 48 hours, or within 72 hours following plasmapheresis. In some embodiments, the method comprises administering intravenous immunoglobulin to the individual during the same treatment session as the plasmapheresis. In some embodiments, the method comprises administering intravenous immunoglobulin to the individual within 24 hours of the plasmapheresis.

In some embodiments, the methods described herein further comprise performing the method of any one of the embodiments described herein on the individual at least two times per month for at least three months, wherein two of the at least two times per month are within the same week of the month. In some embodiments, the methods described herein further comprise performing the plasmapheresis on the individual at least two times per month for at least three months, wherein two of the at least two times per month are within the same week of the month. In some embodiments, the intravenous immunoglobulin is administered to the individual at least two times per month.

In some embodiments, the methods described herein further comprise performing the method of any one of the embodiments described herein on the individual at least one time per month for at least six months. In some embodiments, the methods described herein further comprise performing the plasmapheresis on the individual at least one time per month for at least six months. In some embodiments, intravenous immunoglobulin is administered to the individual at least one or at least two times per month.

In some embodiments, the exogenous particles or the particles is an inorganic particle or an organic particle. In some embodiments, the exogenous particles or the particles are plastic particles. In some embodiments, the organic particle is a polymer particle, a carbon particle, or a combination thereof. In some embodiments, the polymer comprises plastic, polylactic-co-glycolic acid (PLGA), polyacrylonitrile, polystyrene, polyethylene, low-density polyethylene, high-density polyethylene, polypropylene, polyvinyl chloride, polyurethane, and/or polyethylene terephthalate, poly(l-aspartic acid-co-lactic acid), polyethylene glycol, poly(beta-amino ester), polybutyl cyanoacrylate, or chitosan. In some embodiments, the carbon comprises carbon black, graphene oxide, a graphene platelet, a fullerine, a single-walled carbon nanotube, a polycyclic aromatic hydrocarbon, a lipid nanoparticle, or a multi-walled carbon nanotube. In some embodiments, the exogenous particles or the particles comprises particles sized: between 1 nm and 2000 nm; between 1 nm and 1000 nm; between 1 nm and 500 nm; or between 1000 nm and 5 mm.

In some embodiments, the measuring comprises a spectroscopy method. In some embodiments, the measuring comprises a computerized tomography scan, an ultrasound, a positron emission tomography scan, or a magnetic resonance imaging scan. In some embodiments, the measuring comprises flow cytometry, near-infrared spectroscopy (NIR), double shot pyrolysis-gas chromatography/mass spectrometry, Fourier transform infrared (FT-IR) spectrometry, visual inspection with an optical microscope Raman spectroscopy, or surface-enhanced Raman scattering, dynamic light scattering (DLS), or surface plasmon resonance. In some embodiments, the measuring comprises flow cytometry.

Methods disclosed herein may comprise measuring of a level of exogenous particles (e.g., plastic particles or particles) of an individual. The measuring may establish a level of exogenous particles (e.g., plastic particles or particles) in an individual. Methods of measuring a level of exogenous particles (e.g., plastic particles or particles) may comprise a CLIA or a COLA-certified protocol. Methods of measuring a level of exogenous particles (e.g., plastic particles or particles) may comprise determining an amount of exogenous particles (e.g., plastic particles or particles) that is contributed to the measurement from the testing or plasmapheresis materials used (e.g., plastic tubing, plastic tubes, particles in testing media or solutions, particles in plasmapheresis materials or solutions) that is then accounted for in the final measured level of exogenous particles (e.g., plastic particles or particles). For example, a baseline contribution from the testing materials or the plasmapheresis materials may be measured and subtracted from any measurement from a blood sample of an individual.

Methods of measuring a level of exogenous particles (e.g., plastic particles or particles) may comprise sample pre-treatment, physical characterization, or chemical characterization. Sample pre-treatment may comprise chemical digestion of samples, processing diverse human samples including blood, tissues, stools, semen, or urine. Chemicals for digestion may comprise potassium hydroxide (KOH), and may include temperatures ranging from 60-70° C. Physical characterization may comprise optical microscopy to classify particle color, shape, or size. Microscopy methods may comprise stereo microscopes, scanning electron microscopy/energy dispersive spectroscopy (SEM-EDX), or fluorescent microscopes after Nile Red (NR) staining. Chemical characterization may comprise determining polymer composition by Ramn/(μRaman) spectroscopy, fourier transform infrared spectroscopy (FTIR)/micro-FTIR (μFTIR), laser-induced laser direct infrared spectroscopy (LDIR), or chromatography. Chromatography may comprise pyrolysis-gas chromatography mass spectrometry (Py-GC/MS) or high performance liquid chromatography with tandem mass spectrometry (HPLC-MS/MS). Methods of measuring exogenous particles may comprise the use of accoustofluidics to separate exogenous particles from samples. Accoustofluidics may comprise different types of acoustic waves, including surface acoustic waves (SAWs), generated by interdigitated transducers (IDTs). Surface acoustic waves may comprise standing surface acoustic waves (SSAWs) or traveling surface acoustic waves (TSAWs).

In some embodiments, the method further comprises analyzing the exogenous particles or the particles to determine a composition, a size, a distribution of compositions, and/or a distribution of sizes of the exogenous particles or the particles. In some embodiments, the exogenous particles or the particles comprise one or more of a nanobead, a nanofiber, a nanosphere, and/or a nanofragment plastic particle. In some embodiments, the exogenous particles or the particles comprises one or more of a microbead, a microfiber, and/or a microfragment particle. In some embodiments, the method further comprises reducing a risk of depositing a level of the exogenous particles or the particles onto a surface of a blood vessel or in an organ of the individual. In some embodiments, the method further comprises reducing a rate of deposition of the exogenous particles or the particles on a blood vessel or organ of an individual.

In some embodiments, the separating comprises centrifugating the volume of whole blood, passing the volume of whole blood through a semipermeable membrane, or a combination thereof. In some embodiments, the semipermeable membrane comprises pores of between 0.3 μm and 1.5 μm diameter. In some embodiments, the semipermeable membrane comprises pores of between 0.3 μm and 1.0 μm diameter. In some embodiments, the semipermeable membrane comprises pores of about 0.3 μm to about 0.5 μm diameter. In some embodiments, the semipermeable membrane comprises hollow tubes or fibers.

In some embodiments, the exchange fluid comprises one or more therapeutics. In some embodiments, the exchange fluid comprises human serum albumin. In some embodiments, the exchange fluid comprises 5% of the human serum albumin. In some embodiments, the exchange fluid comprises calcium. In some embodiments, the exchange fluid comprises Normal Saline. In some embodiments, the exchange fluid further comprises intravenous immunoglobulin. In some embodiments, the exchange fluid comprises 2 g of the intravenous immunoglobulin.

The methods as described herein may be used in the treatment of a condition or disorder in an individual. As described herein, the methods may be used for treating a condition in an individual in need thereof comprising performing the method of any one of the embodiments as described herein on the individual, thereby treating the condition.

In some embodiments, the condition is associated with exposure to exogenous particles or particles. In some embodiments, the condition is a microplastics burden. A microplastics burden may refer to the accumulation of microscopic plastic particles within biological systems, including the human body. A microplastic burden may be associated with a range of medical conditions, such as gastrointestinal inflammation, respiratory issues, and potential disruption of metabolic and immune functions. A microplastic burden may be associated with oxidative stress, altered gut microbiota, and increased susceptibility to certain chronic diseases. The term microplastics burden may broadly encompass both the presence of microplastics in tissues and their possible contributions to adverse health outcomes.

In some embodiments, the microplastics burden is an inflammatory condition, an autoimmune condition, an endocrine condition, a cardiovascular condition, a reproductive condition, or a neurocognitive condition. In some embodiments, the microplastics burden is associated with deposition of the exogenous particles on a tissue, organ, or cell of the individual. In some embodiments, the tissue is a nerve, vascular, or testicular tissue. In some embodiments, the tissue is a cranial nerve tissue. In some embodiments, the condition is associated with systemic inflammation or localized inflammation. In some embodiments, the localized inflammation is a result of the exogenous particles accumulation at a localized area of inflammation. In some embodiments, the localized area of inflammation is a tissue, organ, or cell of the individual. In some embodiments, the tissue is a nerve, vascular, or testicular tissue. In some embodiments, the condition is an inflammatory, an autoimmune, an endocrine, a cardiovascular, a neurocognitive, or a reproductive disorder.

Also described herein is a method for reducing an exogenous particle level (e.g., a particle level or a plastic particle level) in an individual by carrying out a blood dilution. As described herein, plasma content can be removed and exchange fluid added. The removal of plasma content (even with partial replacement) together with the addition of exchange fluid typically lowers the concentrations of one or more constituents (e.g., exogenous particles, particles, plastics particles, or any combination thereof) of plasma in an individual receiving plasmapheresis treatment. Essentially, solute (the plasma content) is decreased while the solvent (the liquid portion of plasma replaced by the exchange fluid) remains the same. In this way, in addition to removing plasma content components, the methods described herein dilute one or more components of plasma content that remain in the blood of the individual receiving plasmapheresis following a plasmapheresis treatment.

Plasma content dilution can achieve synergistic affects to removal of plasma content (e.g., exogenous particles, plastic particles, particles, or any combination thereof) and the dilution may cause the plasma composition of an individual receiving plasmapheresis to have a reduced level of exogenous particles, plastic particles, particles, or any combination thereof. The decrease in concentration of the plasma content (e.g., exogenous particles, plastic particles, particles, or any combination thereof) in individuals receiving plasmapheresis as described herein may lower the risk for any condition associated with a microplastics burden (e.g., an inflammatory condition, an autoimmune condition, an endocrine condition, or any combination thereof).

A number of models may be used to calculate a level of dilution that is achieved with a plasmapheresis treatment as described herein. For example, Reverberi and Reverberi (Blood Transfus, 2007; 5 (3): 164-174) provide formulas for modeling residual plasma analyte concentrations from which Formulas (I), (Ia), and (Ib) are derived:

$$Residual Solute Concentration = 100 \% * {e}^{- \frac{{v}_{p} * n}{{v}_{b}}} \qquad ( I )$$

wherein vₚ is plasma volume, n is number of plasma volume units exchanged, and v_{b} is total blood volume (e.g., whole blood, or peripheral blood volume).

As an illustrative example of Formula (I), using a plasma volume of 3 L, 1 plasma volume to be exchanged, and a total volume of 5 L of whole blood would be expected to result in a residual plasma content concentration of approximately 54% of the original concentration following a single plasmapheresis treatment. For the same plasma volume and total volume of blood but a plasma exchange of 1.5 plasma volumes, the residual solute concentration is approximately 40%.

Plasma analytes exhibit an instantaneous concentration drop following blood exchange, followed by reconcentration along a logarithmic-like trend to pre-blood dilution levels. Formula (I) can be modified to Formula (Ia) to estimate plasma analyte levels n_{d} days following plasma exchange, assuming a half-time t_{1/2} for the analytes to return to pre-blood dilution levels:

$$Residual Solute Concentration = 100 \% * ( 1 - {( \frac{1}{2} )}^{\frac{{n}_{d}}{{t}_{\frac{1}{2}}}} {e}^{- \frac{{v}_{p} * n}{{v}_{b}}} ) . \qquad ( IA )$$

Noting that not all plasma analytes reestablish homeostasis at equal rates, as typical plasma analytes return to pre-blood dilution levels after about 10 days, t_{1/2} of 3 to 4 days is often a suitable estimate. Once again using 3 L, 1 plasma volume, and 5 L of whole blood with a half-life of 3 days and measured 3 days after the initial plasmapheresis treatment, the residual solute concentration increases from approximately 54% following the plasmapheresis treatment to approximately 72% three days (i.e., 72 hours) later.

Winters (cited above) provides the following simplified formula: Y/Y0=e^{-x}, where Y is the final concentration of a substance, Y0 is the initial concentration, and X is the number of times the patient's plasma volume is exchanged. Continuing with the approximately 72% residual concentration calculated above using Formula (Ia), if plasmapheresis is administered once again 72 hours after the initial plasmapheresis, Y0=72%, x=1, and Y=26.5% residual plasma concentration following the second plasmapheresis treatment.

Formula (Ia) can further be used to determine the degree of plasma analyte clearance following multiple rounds of plasma exchanges by setting n as above. For such an application, Formula (Ia) can be estimated as a sum, expressed as Formula (Ib), where a plasma exchange event, i, is attenuated by analyte regeneration over n_{di} days following the plasma exchange event:

$$Residual Solute Concentration = 100 \% * ( 1 - {\sum }_{i} {( \frac{1}{2} )}^{\frac{{n}_{di}}{{t}_{\frac{1}{2}}}} {e}^{- \frac{{v}_{p}}{{v}_{b}}} ) . \qquad ( IB )$$

This equation does not account for diminished plasma exchange efficacy over multiple cycles, reflecting exchange of partially diluted plasma. However, in embodiments where t_{1/2} is approximately equal to or less than the time between plasma exchange events, and for therapies with limited numbers of plasma exchanges, discrepancies from this estimation are typically small.

Formulas (I), (Ia), (Ib), and the formula provided by Winters provide excellent and useful approximations of dilution levels which are suitable for use with the methods described herein. It is also noted that if higher accuracy in calculating plasma content dilution is desired, the following factors and issues should also be considered: First, many plasma separation methods partially separate plasma from cellular components. While multiple rounds of separation can increase efficiencies, single iterations of centrifuge-based plasma separation and membrane-based plasma separation typically achieve 80% and 30% plasma separation efficiencies, respectively (Williams and Balogun. (Clin J Am Soc Nephrol, 2014; 9 (1): 181-190). For a 500 mL blood draw, which will typically contain about 55% (275 mL) plasma, these efficiencies translate to 220 mL plasma removed by centrifugation and 82.5 mL plasma removed by filtration. The effect of incomplete plasma separation from cellular components is a concomitant decrease in plasma exchange efficiency. If plasma is centrifugally separated from blood with 80% efficiency, then plasma exchange will typically be 80% efficient given a volume of blood separated and exchanged. Second, many plasma components actively exchange into spaces outside of the vasculature. While a typical adult male human has about 5 liters of blood, the majority of fluids, and analogously, analytes, are contained in interstitial and intracellular spaces, which account for about 10.5 and 28 liters of fluid, respectively. As used herein, intracellular spaces can include volumes contained within cell membranes of an organism, including fluids within these spaces, while interstitial spaces can denote spaces surrounding tissues. Many plasma analytes actively equilibrate between the blood and these spaces, such that fractions of their total populations are contained within the blood at any given time. Removal of such species by plasma exchange can be attenuated by their partitioning outside of the blood. For example, about 60% to 70% of IgG1 immunoglobulins are present in the blood at any given time, such that plasma exchange can target 60% to 70% of the IgG1 population. Furthermore, disruption of homeostasis, for example through blood dilution, can affect osmotic gradients which draw species into blood from extravascular spaces, thereby hastening return to pre-treatment blood analyte levels. Third, plasma analytes regenerate at a range of rates. Following a blood composition altering event, such as blood dilution, blood analyte levels tend to return to their original, resting levels. Although blood dilution can alter resting levels of individual blood analytes (for example lowered blood triglyceride levels as outlined in Dehal and Adashek. Case Rep Med, 2018; 2018:4017573), diluted species tend to increase in concentration while concentrated species (e.g., albumin provided from a high concentration exchange fluid) tend to decrease in concentration to reestablish pre-dilution levels. While some species (in particular many cytokines) exhibit complex re-equilibration patterns, many follow simple exponential growth or decay curves. However, the rates of these processes can vary significantly between species. For example, IgG immunoglobulins often return to pre-blood dilution levels in about 4 days (Harris et al., Journal of Scleroderma and Related Disorders, 2018; 3 (2): 132-152), while low-density lipoproteins can take more than 2 weeks to return to resting levels (McGowan. Journal of Clinical Lipidology, 2013; 7 (3): S21-S26).

May et al., (Am J Clin Pathol, 1989; 91 (6): 688-94) presents a model which accounts for extravascular compartmentalization and species regeneration. This model, adapted as Formula (II) below, captures rates of plasma analyte loss through plasma exchange and clearance:

$${A}_{t} = {A}_{0} * {e}^{[ - \frac{( r + {k}_{1} ) t}{{V}_{p}} ]} + \frac{{V}_{p} {k}_{2} [ 1 - {e}^{[ - \frac{( r + {k}_{1} ) t}{{V}_{p}} ]} ]}{r + {k}_{1}} , \qquad ( II )$$

wherein Aₜ denotes a plasma analyte concentration at time t, A₀ denotes the initial concentration of the analyte, k₁ denotes a rate of clearance of the analyte (e.g., through degradation, biliary clearance, etc.), and k₂ denotes a rate at which the analyte is released from extracellular tissues. The May et al. model does not account for regeneration of the analyte, and further assumes plasma analyte distribution according to a two-compartment system (intravascular and extravascular). Accordingly, the model may not be appropriate for analytes dynamically exchanged across multiple compartments (e.g., antibodies with appreciable endosomal and interstitial concentrations), or analytes which are rapidly produced following dilution. However, this model can be corrected for inefficient plasma removal during exchange, as outlined above, through correction to clearance rate (k₁).

In some embodiments, removal of particles from blood at the described rates with the described methods leads to improvement in oxidative and inflammatory intestinal symptoms, improvement in gut epithelial permeability, reduction in oxidative stress, increased energy, improvement in fertility, reduction of inflammation, and improvement in microbiota community structure. Removal of particles also removes adsorbed chemical additives and biofilms associated with the particles, improving health outcomes.

A number of implementations have been described. Nevertheless, it will be understood that various modifications may be made without departing from the spirit and scope of the disclosure. Accordingly, other implementations are within the scope of the following claims.

### NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.
1. A method of reducing a level of particles in an individual by plasmapheresis, the method comprising: (a) withdrawing a volume of whole blood from the individual; (b) separating the volume of whole blood into a cellular fraction and a particle fraction, wherein the particle fraction comprises a level of particles; and (c) returning the cellular fraction and an exchange fluid to the circulatory system of the individual, thereby reducing the level of particles in the individual. 2. The method of embodiment 1, wherein the particle is an inorganic particle or an organic particle. 3. The method of any one of embodiments 1-2, wherein the particles are plastic particles and the particle fraction is a plastic particle fraction comprising a plurality of plastic particles. 4. The method of embodiment 1, further comprising reducing a level of particles in the circulatory system of the individual. 5. The method of any one of embodiments 1or 4, further comprising reducing a level of particles in an organ of the individual. 6. The method of any one of embodiments 1-5, further comprising reducing a level of particles on a blood vessel wall of the individual. 7. The method of embodiment 2, wherein the inorganic particle comprises bismuth oxide (Bi₂O₃), silicon dioxide (SiO₂), copper oxide (CuO), zinc oxide (ZnO), titanium dioxide (TiO₂), silver (Ag), gold (Au), platinum (Pt), iron oxide (Fe₂O₃), cerium oxide (CeO₂), cobalt oxide (Co₃O₄), aluminum oxide (Al₂O₃), molybdenum trioxide (MoO₃), magnesium oxide (MgO), nickel oxide (NiO), chromium oxide (Cr₂O₃), tungsten oxide (WO₃), yttrium oxide (Y₂O₃), terbium-doped gadolinium, gold, silver, platinum, nitrate, or manganese oxide (Mn₂O₃). 8. The method of embodiment 2, wherein the organic particle is a polymer particle, a carbon particle, or a combination thereof. 9. The method of embodiment 8, wherein the polymer comprises plastic, polylactic-co-glycolic acid (PLGA), polyacrylonitrile, polystyrene, polyethylene, low-density polyethylene, high-density polyethylene, polypropylene, polystyrene, polyvinyl chloride, polyurethane, and/or polyethylene terephthalate, poly(l-aspartic acid-co-lactic acid), polyethylene glycol, poly (beta-amino ester), polybutyl cyanoacrylate, polyethylene terephthalate, or chitosan. 10. The method of embodiment 8, wherein the carbon comprises carbon black, graphene oxide, a graphene platelet, a fullerine, a single-walled carbon nanotube, a polycyclic aromatic hydrocarbon, a lipid nanoparticle, or a multi-walled carbon nanotube. 11. The method of embodiment 8, wherein the level of particles comprises particles sized: between 1 nm and 2000 nm; between 1 nm and 1000 nm; between 1 nm and 500 nm; or between 1000 nm and 5 mm. 12. The method of any one of embodiments 1-11, comprising repeating steps (a) through (c) until the level of particles in one or more of the following is below a threshold level: the circulatory system of the individual; the organ of the individual; and/or the blood vessel wall of the individual. 13. The method of any one of embodiments 1-12, further comprising measuring a level of particles in the circulatory system of the individual. 14. The method of any one of embodiments 1-13, further comprising measuring a level of particles in the organ of the individual 15. The method of any one of embodiments 1-14, further comprising measuring a level of particles in the blood vessel of the individual. 16. The method of any one of embodiments 13-15, wherein the measuring is performed before withdrawing the volume of whole blood to yield a pretreatment level of particles. 17. The method of any one of embodiments 13-16, wherein the measuring is performed after returning the cellular fraction and the exchange fluid to the circulatory system of the individual to yield a post-treatment level of particles. 18. The method of any one of embodiments 13-17, wherein the measuring is performed in real-time during one or more of steps (a) through (c). 19. The method of any one of embodiments 1-18, further comprising repeating steps (a) through (c) a plurality of times. 20. The method of any one of embodiments 1-19, comprising repeating steps (a) through (c) until the level of particles in the individual reaches a target level. 21. The method of embodiment 20, wherein the target level is not greater than 500 particles/μL in the circulatory system of the individual. 22. The method of any one of embodiments 20-21, wherein the target level is between 100 and 500 particles/μL in the circulatory system of the individual. 23. The method of embodiment 20, wherein the target level is 90%, 80%, 70%, 60%, 50%, 40%, 30%, 20%, 10%, or 1% of the pretreatment level. 24. The method of any one of embodiments 13-23, wherein the measuring is performed by an imaging method. 25. The method of any one of embodiments 13-24 wherein the measuring is performed using a computerized tomography scan, an ultrasound, a positron emission tomography scan, or a magnetic resonance imaging scan. 26. The method of any one of embodiments 13-25, wherein the measuring is performed using flow cytometry, near-infrared spectroscopy (NIR), double shot pyrolysis-gas chromatography/mass spectrometry, Fourier transform infrared (FT-IR) spectrometry, visual inspection with an optical microscope Raman spectroscopy, or surface-enhanced Raman scattering, dynamic light scattering (DLS), or surface plasmon resonance. 27. The method of any one of embodiments 13-26, wherein the measuring is performed using flow cytometry. 28. The method of any one of embodiments 13-27, wherein the particle is a plastic particle; and wherein the measuring comprises contacting the plastic particle with a fluorescent dye. 29. The method of embodiment 28, wherein the fluorescent dye is a lipophilic dye or a phenoxazone dye. 30. The method of embodiment 28 or 29, wherein the fluorescent dye is Nile Red, BODIPY™ 493/503, Rhodamine B, or a combination thereof. 31. The method of any one of embodiments 13-24, wherein the measuring comprises using an antibody specific for a particle. 32. The method of any one of embodiments 1-31, wherein the exchange fluid comprises an immunoglobulin. 33. The method of embodiment 32, wherein the immunoglobulin comprises IVIG. 34. The method of embodiment 33, wherein the IVIG has a concentration of between 0.5 g/kg and 2 g/kg of bodyweight of the individual. 35. The method of any one of embodiments 1-34, further comprising repeating the method once per week, twice per week, three times a week, twice a week, weekly, biweekly, or monthly. 36.The method of any one of embodiments 1-35, wherein the method is repeated over a period of at least one month, at least two months, at least three months, at least four months, at least five months, at least six months, or greater than six months. 37. The method of any one of embodiments 1-36, further comprising analyzing the level of particles to determine a composition, a size, a distribution of compositions, and/or a distribution of sizes of the particles in the level of particles. 38. The method of any one of embodiments 1-37, wherein the level of particles comprises one or more of a nanobead, a nanofiber, a nanosphere, and/or a nanofragment plastic particle. 39. The method of any one of embodiments 1-37, wherein the level of particles comprises one or more of a microbead, a microfiber, and/or a microfragment particle. 40. The method of any one of embodiments 1-39, further comprising reducing a risk of depositing a level of particles onto a surface of a blood vessel or in an organ of the individual. 41. The method of any one of embodiments 1-40, further comprising reducing a rate of deposition of particles on a blood vessel or organ of an individual. 42. The method of any one of embodiments 1-41, wherein the separating comprises centrifugating the volume of whole blood, passing the volume of whole blood through a semipermeable membrane, or a combination thereof. 43. The method of embodiment 42, wherein the semipermeable membrane comprises pores of between 0.3 μm and 1.5 μm diameter. 44. The method of embodiment 42, wherein the semipermeable membrane comprises pores of between 0.3 μm and 1.0 μm diameter. 45. The method of embodiment 42, wherein the semipermeable membrane comprises pores of about 0.3 μm to about 0.5 μm diameter. 46. The method of any one of embodiments 42-45, wherein the semipermeable membrane comprises hollow tubes or fibers. 47. The method of any one of embodiments 13-46, wherein the measuring comprises binding a particle with an antibody complex comprising a detectable agent. 48. The method of any one of embodiments 13-46, wherein the measuring comprises binding a particle with an aptamer complex comprising a detectable agent. 49. The method of any one of embodiments 47-48, wherein the detectable agent is a fluorescent dye. 50. A system for reducing a level of particles in an individual, the system comprising: a plasmapheresis unit configured to separate particles from whole blood; and a measuring unit configured to analyze a sample of particles. 51. The system of embodiment 50, wherein the system is configured to perform the method of any one of embodiments 1-45. 52. The system of any one of embodiments 50-51 further comprising a processor for operating the plasmapheresis unit and/or measuring unit. 53. The system of any one of embodiments 50-52, wherein the measuring unit is a computer tomography scanner, an ultrasound machine, a magnetic resonance imaging scanner, a flow cytometer, an immunoassay, a mass spectrometer, a Raman spectrometer, a surface plasmon resonance instrument, or a Fourier Transform Infrared (FT-IR) spectrometer. 54. The system of any one of embodiments 50-53, wherein the measuring unit is a flow cytometer. 55. The system of embodiment 54, wherein the flow cytometer comprises a violet side scatter unit. 56. The system of any one of embodiments 50-55, further comprising a display. 57. The system of embodiment 56, wherein the display is configured to show real-time particle levels in withdrawn whole blood. 58. The system of any one of embodiments 52-57, wherein the processor is further configured to depict a particle level, a target particle level, or a combination thereof. 59. The system of any one of embodiments 50-58, wherein the system is automated. 60. The system of any one of embodiments 50-59, further comprising blood collection tubes comprising polyethylene terephthalate. 61. The system of any one of embodiments 50-60, further comprising a centrifugal blood separation unit. 62. The system of any one of embodiments 50-61, further comprising a semipermeable membrane. 63. The system of any one of embodiments 50-62, further comprising a coating configured to adsorb a particle. 64. The system of embodiment 63, wherein the coating comprises a polystyrene sulfonate or a polyaspartate. 65. The system of any one of embodiments 50-64, further comprising a plastic-specific adsorption column.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Pilot Study for Plastic Particle Reduction Using Therapeutic Plasma Exchange

This example describes the results of a human pilot study using therapeutic plasma exchange (TPE) for the removal of plastic particles from patients.

Several patients were enrolled in a pilot study in which the levels of plastic particles in the plasma were evaluated prior to (pre-TPE) and after (post-TPE) administering therapeutic plasma exchange (TPE). Blood samples were taken from each of the patients before and after TPE by blood collection using a blood draw line, a collection tube and pipette, or a lancet. During TPE, the patient's blood was passed through an apheresis machine, where the filtered plasma was removed and discarded, and then the filtered red blood cells, white blood cells, and platelets, were combined with an exchange fluid, comprising Normal Saline and 5% human serum albumin and calcium, which was then returned to the patient's circulation at the same volume of the volume of plasma that was removed. Optionally, the patients were also then administered 2 g of IVIG.

Plastic particles were quantified and analyzed in pre and post TPE patient blood samples by application of a dye that specifically stains plastic particles in the blood sample. Using imaging techniques, the plastic particles (measured ranging in size from 1-70 μm) were detected and counted in each of the blood samples.

TABLE 1 below provides the quantitative results of plastics testing from blood samples collected from each patient prior to (“pre-TPE”) and after (“post-TPE”) undergoing therapeutic plasma exchange (TPE). TABLE 1 also provides the measured reduction in plastic particles for a given patient by the difference between the measured plastic particles in the patient blood samples from the pre-TPE sample and the post-TPE sample. A positive difference means a reduction in plastic particles and a negative difference means an increase in plastic particles from the pre-TPE to post-TPE blood samples.

TABLE 1
Pre- and Post-TPE Levels of Plastic
Particles in Patient Blood Samples

| | Plastic Particles | Plastic Particles | Reduction in Plastic |
|---|---|---|---|
| Sample | Pre-TPE | Post-TPE | Particles |
| Patient 1 | 154 | 54 | 100 |
| Patient 2 | 123 | 37 | 86 |
| Patient 3 | 95 | 21 | 74 |
| Patient 4 | 40 | 2 | 38 |
| Patient 5 | 46 | 13 | 33 |
| Patient 6 | 43 | 12 | 31 |
| Patient 7 | 40 | 9 | 31 |
| Patient 8 | 56 | 29 | 27 |
| Patient 9 | 44 | 18 | 26 |
| Patient 10 | 37 | 12 | 25 |
| Patient 11 | 29 | 6 | 23 |
| Patient 12 | 24 | 1 | 23 |
| Patient 13 | 26 | 5 | 21 |
| Patient 14 | 21 | 0 | 21 |
| Patient 15 | 38 | 18 | 20 |
| Patient 16 | 30 | 11 | 19 |
| Patient 17 | 22 | 3 | 19 |
| Patient 18 | 27 | 10 | 17 |
| Patient 19 | 38 | 22 | 16 |
| Patient 20 | 32 | 16 | 16 |
| Patient 21 | 19 | 3 | 16 |
| Patient 22 | 74 | 59 | 15 |
| Patient 23 | 20 | 7 | 13 |
| Patient 24 | 22 | 11 | 11 |
| Patient 25 | 12 | 1 | 11 |
| Patient 26 | 18 | 8 | 10 |
| Patient 27 | 11 | 1 | 10 |
| Patient 28 | 12 | 3 | 9 |
| Patient 29 | 11 | 3 | 8 |
| Patient 30 | 9 | 1 | 8 |
| Patient 31 | 8 | 0 | 8 |
| Patient 32 | 30 | 23 | 7 |
| Patient 33 | 21 | 14 | 7 |
| Patient 34 | 8 | 1 | 7 |
| Patient 35 | 7 | 0 | 7 |
| Patient 36 | 7 | 1 | 6 |
| Patient 37 | 13 | 9 | 4 |
| Patient 38 | 6 | 2 | 4 |
| Patient 39 | 6 | 2 | 4 |
| Patient 40 | 5 | 1 | 4 |
| Patient 41 | 28 | 25 | 3 |
| Patient 42 | 18 | 15 | 3 |
| Patient 43 | 10 | 7 | 3 |
| Patient 44 | 7 | 4 | 3 |
| Patient 45 | 30 | 28 | 2 |
| Patient 46 | 14 | 12 | 2 |
| Patient 47 | 14 | 12 | 2 |
| Patient 48 | 8 | 6 | 2 |
| Patient 49 | 7 | 5 | 2 |
| Patient 50 | 2 | 0 | 2 |
| Patient 51 | 60 | 59 | 1 |
| Patient 52 | 4 | 3 | 1 |
| Patient 53 | 4 | 3 | 1 |
| Patient 54 | 16 | 16 | 0 |
| Patient 55 | 3 | 3 | 0 |
| Patient 56 | 1 | 1 | 0 |
| Patient 57 | 14 | 15 | −1 |
| Patient 58 | 8 | 9 | −1 |
| Patient 59 | 6 | 7 | −1 |
| Patient 60 | 3 | 4 | −1 |
| Patient 61 | 2 | 3 | −1 |
| Patient 62 | 9 | 11 | −2 |
| Patient 63 | 8 | 10 | −2 |
| Patient 64 | 7 | 9 | −2 |
| Patient 65 | 5 | 7 | −2 |
| Patient 66 | 1 | 3 | −2 |
| Patient 67 | 45 | 48 | −3 |
| Patient 68 | 7 | 10 | −3 |
| Patient 69 | 6 | 9 | −3 |
| Patient 70 | 4 | 7 | −3 |
| Patient 71 | 3 | 6 | −3 |
| Patient 72 | 1 | 4 | −3 |
| Patient 73 | 1 | 4 | −3 |
| Patient 74 | 0 | 3 | −3 |
| Patient 75 | 4 | 8 | −4 |
| Patient 76 | 3 | 7 | −4 |
| Patient 77 | 1 | 5 | −4 |
| Patient 78 | 0 | 4 | −4 |
| Patient 79 | 26 | 31 | −5 |
| Patient 80 | 22 | 27 | −5 |
| Patient 81 | 11 | 16 | −5 |
| Patient 82 | 9 | 14 | −5 |
| Patient 83 | 4 | 9 | −5 |
| Patient 84 | 4 | 9 | −5 |
| Patient 85 | 3 | 8 | −5 |
| Patient 86 | 2 | 7 | −5 |
| Patient 87 | 1 | 6 | −5 |
| Patient 88 | 1 | 6 | −5 |
| Patient 89 | 0 | 5 | −5 |
| Patient 90 | 19 | 25 | −6 |
| Patient 91 | 12 | 19 | −7 |
| Patient 92 | 9 | 16 | −7 |
| Patient 93 | 6 | 13 | −7 |
| Patient 94 | 5 | 12 | −7 |
| Patient 95 | 2 | 9 | −7 |
| Patient 96 | 1 | 8 | −7 |
| Patient 97 | 7 | 15 | −8 |
| Patient 98 | 5 | 13 | −8 |
| Patient 99 | 0 | 8 | −8 |
| Patient 100 | 6 | 15 | −9 |
| Patient 101 | 4 | 13 | −9 |
| Patient 102 | 13 | 23 | −10 |
| Patient 103 | 13 | 23 | −10 |
| Patient 104 | 12 | 22 | −10 |
| Patient 105 | 4 | 16 | −12 |
| Patient 106 | 21 | 34 | −13 |
| Patient 107 | 12 | 25 | −13 |
| Patient 108 | 6 | 21 | −15 |
| Patient 109 | 9 | 25 | −16 |
| Patient 110 | 4 | 20 | −16 |
| Patient 111 | 21 | 38 | −17 |
| Patient 112 | 5 | 22 | −17 |
| Patient 113 | 4 | 21 | −17 |
| Patient 114 | 0 | 17 | −17 |
| Patient 115 | 22 | 40 | −18 |
| Patient 116 | 7 | 25 | −18 |
| Patient 117 | 4 | 24 | −20 |
| Patient 118 | 0 | 21 | −21 |
| Patient 119 | 17 | 42 | −25 |
| Patient 120 | 12 | 37 | −25 |
| Patient 121 | 7 | 32 | −25 |
| Patient 122 | 4 | 29 | −25 |
| Patient 123 | 2 | 27 | −25 |
| Patient 124 | 0 | 25 | −25 |
| Patient 125 | 0 | 26 | −26 |
| Patient 126 | 22 | 54 | −32 |
| Patient 127 | 9 | 41 | −32 |
| Patient 128 | 3 | 40 | −37 |
| Patient 129 | 14 | 52 | −38 |
| Patient 130 | 1 | 41 | −40 |
| Patient 131 | 5 | 50 | −45 |
| Patient 132 | 7 | 53 | −46 |
| Patient 133 | 5 | 52 | −47 |
| Patient 134 | 7 | 55 | −48 |
| Patient 135 | 5 | 56 | −51 |
| Patient 136 | 0 | 52 | −52 |
| Patient 137 | 29 | 94 | −65 |

Taken together, the results of TABLE 1 show a mean reduction of 2.2 plastic particles and a median reduction of 3 plastic particles in patient blood samples following TPE. The standard deviation was 22.4 plastic particles, with a maximum reduction of 100 plastic particles following TPE, and a maximum increase of 65 plastic particles following TPE.

As shown in the results in TABLE 1, 53 out of the 137 patients showed a reduction in plastic particles in their blood after TPE while the other 84 of the 137 patients showed an increase in plastic particles in their blood after TPE. Without being bound by a particular theory, it is thought that patients arriving in the clinics with higher numbers of plastic particles in their bloodstream, TPE may provide a greater therapeutic benefit (e.g., may reduce the plastic particles to a greater extent).

For example, patients with greater levels of plastic in their blood (e.g., 30 plastic particles and above) TPE were seen to provide a greater therapeutic benefit. For the 19 patients in TABLE 1 with equal to or greater than 30 plastic particles in their pre-TPE blood sample, the average pretreatment particle count decreased from 55.52 to 25.8 particles in this patient group after TPE, yielding an average reduction of 53.53% plastic particles following TPE.

As described in this example, TPE may offer a therapeutic benefit for reducing plastic particles in the bloodstream of a patient.

### Example 2

### Removal of Plastic Particles from the Circulatory System

This example demonstrates the use of plasmapheresis for removing plastic particles from an individual's circulatory system.

A patient presents with concerns about their exposure to environmental pollutants, particularly plastic particles. A blood sample is taken from the patient, which undergoes initial testing with flow cytometry using Nile Red dye to quantitate plastic particle levels in the patient's circulatory system. The testing reveals 1200 particles/μL of plastic particles in their blood. Based on these measured levels, the patient is provided a program of weekly plasmapheresis treatments for three months to remove a portion of the plastic particles in the patient's circulatory system and improve the patient's overall health.

The patient undergoes their first plasmapheresis session. During the procedure, whole blood or peripheral blood is withdrawn and separated into a cellular fraction and a plastic particle fraction comprising plastic particles. The plastic particle fraction comprising the plastic particles is removed and replaced with an exchange fluid comprising albumin and immunoglobulins. The cellular fraction is then returned to the circulatory system of the patient along with the exchange fluid. After the session, a total of one and a half volumes of plasma has been removed.

Throughout the treatment, the removed plastic particle fraction is analyzed in real-time using a flow cytometer equipped with Nile Red labeling capabilities. This allows for continuous monitoring of the plastic levels being removed from the patient's bloodstream. A steady decrease is observed in the plastic particle levels in the patient's circulatory system as the treatment progresses.

After the first session, a post-treatment blood sample is taken and analyzed. The results show a significant reduction in plastic particle levels post-treatment compared to the pre-treatment sample. Additional plasmapheresis is performed to achieve a target plastic particle level.

The patient undergoes a total of twelve plasmapheresis cycles over the course of three months. During each cycle, the plastic particle levels are monitored in real-time, and post-treatment blood samples are analyzed. A consistent decrease in plastic particle levels is observed with each successive treatment.

After the eighth cycle, the blood analysis reveals that the plastic particle levels in the patient's circulatory system have fallen below the predetermined target levels. Below-target plastic particle levels are maintained through regular additional plasmapheresis treatments during each of which plastic particle fractions are removed.

### Example 3

### Removal of Plastic Particles from Colon

This example demonstrates the effectiveness of plasmapheresis in removing plastic particles from an individual's tissue, including the patient's colon.

A patient presents with concerns about their exposure to environmental pollutants, particularly plastic particles. A colon biopsy is taken from the patient, which undergoes initial testing with flow cytometry using Nile Red dye to assess plastic particle levels. The testing reveals a level of 2 particles/μg of plastic particles in the patient's colon, compared with an average individual, who is estimated to have approximately 0.03 particles/μg. The patient undergoes biweekly plasmapheresis treatments for six months to remove the plastic particles and improve the patient's overall health.

The patient undergoes a first plasmapheresis session. During the procedure, whole blood or peripheral blood is withdrawn and separated into a cellular fraction and a plastic particle fraction comprising plastic particles. The plastic particle fraction comprising the plastic particles is removed and replaced with an exchange fluid comprising albumin and immunoglobulins. The cellular fraction is then returned to the patient's circulatory system of the patient along with the exchange fluid. After the session, a total of 1.5 volumes of plasma has been removed.

Throughout the treatment, the removed plastic particle fraction is analyzed in real-time using a flow cytometer equipped with Nile Red labeling capabilities. This allows for continuous monitoring of the plastic particle levels in the patient's bloodstream. A steady decrease in plastic particle level is observed as the treatment progresses.

After the first session, a post-treatment blood sample is taken and analyzed. The results show a significant reduction in plastic levels of the blood compared to a pre-treatment sample of blood. A reduction in plastic particle levels in the circulatory system is correlated with reduced plastic particle levels in the colon. Additional plasmapheresis sessions are utilized to maintain plastic particle levels in the colon at or below a target level.

The patient undergoes a total of 18 plasmapheresis sessions over the course of six months. During each session, the plastic particle levels in the blood are monitored in real-time, and post-treatment blood samples are analyzed to quantitate plastic particle levels. A decrease in plastic particle levels of the blood are observed with each successive treatment.

After the eighth session, a further colon biopsy reveals that the plastic particle levels in the colon tissue have dropped significantly and below the target level. Target plastic particle levels in the colon are maintained through regular additional plasmapheresis treatments during each of which plastic particle fractions are removed.

### Example 4

### Removal of Plastic Particles from the Liver

This example demonstrates the effectiveness of plasmapheresis in removing plastic particles from an individual's tissue, including the patient's liver.

A patient presents with concerns about their exposure to environmental pollutants, particularly plastic particles. An ultrasound scan is taken from the patient. The testing reveals 2 particles/μg of plastic particles in the liver. Compared with an average individual, who is estimated to have approximately 0.03 particles/μg in the liver. The patient undergoes biweekly plasmapheresis treatments for six months to remove the plastic particles and improve the patient's overall health.

The patient undergoes a first plasmapheresis session. During the procedure, whole blood or peripheral blood is withdrawn and separated into a cellular fraction and a plastic particle fraction comprising plastic particles. The plastic particle fraction comprising the plastic particles is removed and replaced with an exchange fluid comprising albumin and immunoglobulins. The cellular fraction is then returned to the patient's circulatory system of the patient along with the exchange fluid. After the session, a total of 2 volumes of plasma has been removed.

Throughout the treatment, the removed plastic particle fraction is analyzed in real-time using a flow cytometer equipped with Nile Red labeling capabilities. This allows for continuous monitoring of the plastic particle levels in the patient's bloodstream. A steady decrease in plastic particle levels is observed as the treatment progresses.

After the first session, a post-treatment blood sample is taken and analyzed. The results show a significant reduction in plastic levels of the blood compared to a pre-treatment sample of blood. A reduction in plastic particle levels in the circulatory system are correlated with reduced plastic particle levels in the liver. Additional plasmapheresis sessions can be utilized to maintain plastic particle levels in the liver at or below a target level.

The patient undergoes a total of 16 plasmapheresis sessions over the course of four months. During each session, the plastic particle levels in the blood are monitored in real-time, and post-treatment blood samples are analyzed to quantitate plastic particle levels. A decrease in plastic particles of the blood is observed with each successive treatment.

After the fourth session, further ultrasound imaging of the liver reveals that the plastic particle levels in the liver tissue have dropped significantly and below the target level. Target plastic particle levels in the liver are maintained through regular additional plasmapheresis treatments during each of which plastic particle fractions are removed.

### General Statements Regarding this Disclosure

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. For example, the methods described herein do not require the particular order of steps illustrated to achieve the desired result but rather one or more of the steps of a method as described herein may be performed in a different order with respect to the other steps. Moreover, additional steps or operations may be provided or steps or operations may be eliminated to achieve the desired result.

It will be understood by one of ordinary skill in the art that all or a portion of the methods disclosed herein may be embodied in a non-transitory machine readable or accessible medium comprising instructions readable or executable by a processor or processing unit of a computing device or other type of machine.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein.

All existing subject matter mentioned herein (e.g., publications, patents, patent

applications and hardware) is incorporated by reference herein in its entirety except insofar as the subject matter may conflict with that of the present invention (in which case what is present herein shall prevail). The referenced items are provided solely for their disclosure prior to the filing date of the present application.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

This disclosure is not intended to be limited to the scope of the particular forms set forth but is intended to cover alternatives, modifications, and equivalents of the variations or embodiments described herein. Further, the scope of the disclosure fully encompasses other variations or embodiments that may become obvious to those skilled in the art in view of this disclosure.

What is claimed is:

1. A method of reducing a level of exogenous particles in an individual by plasmapheresis, the method comprising:
(a) withdrawing a volume of whole blood of the individual having a pre-treatment level of exogenous particles;
(b) administering the plasmapheresis to the individual, thereby reducing the level of exogenous particles in the individual; and
(c) repeating steps (a) and (b) until a post-treatment level of exogenous particles in the individual reaches a target value that is at least 10% less than the pre-treatment level of exogenous particles.

2. The method of claim 1, wherein the target value is at least 20% less than the pre-treatment level of exogenous particles.

3. The method of claim 1, wherein the target value is at least 40% less than the pre-treatment level of exogenous particles.

4. The method of claim 1, wherein the target value is at least 50% less than the pre-treatment level of exogenous particles.

5. The method of claim 1, wherein the target value is less than 50% of the pre-treatment level of exogenous particles in a circulatory system of the individual.

6. The method of claim 1, wherein the target value is less than 50% of the pre-treatment level of exogenous particles in a blood vessel wall of the individual.

7. The method of claim 1, wherein the target value is less than 50% of the pre-treatment level of exogenous particles in an organ of the individual.

8. The method of claim 1, further comprising determining a quantitative reduction from the pre-treatment level of exogenous particles to the post-treatment level of exogenous particles in the individual.

9. The method of claim 1, wherein the target value is an exogenous particle level in a whole blood of the individual.

10. The method of claim 1, wherein the target value is an exogenous particle level in a circulatory system of the individual.

11. The method of claim 1, wherein the target value is an exogenous particle level in an organ, a tissue, or a cell of the individual.

12. The method of claim 11, wherein the tissue is a nerve tissue, a vascular tissue, or a testicular tissue.

13. The method of claim 11, wherein the tissue is a cranial nerve tissue.

14. The method of claim 1, wherein administering the plasmapheresis comprises exchanging at least one unit of plasma volume.

15. The method of claim 1, wherein repeating steps (a) and (b) occurs over a plurality of treatment sessions.

16. The method of claim 1, wherein repeating steps (a) and (b) occurs over a single treatment session.

17. The method of claim 1, further comprising administering intravenous immunoglobulin to the individual.

18. The method of claim 1, further comprising administering intravenous immunoglobulin to the individual following step (b).

19. The method of claim 1, further comprising administering intravenous immunoglobulin to the individual during the same treatment session as administering the plasmapheresis.

20. The method of claim 1, further comprising administering intravenous immunoglobulin to the individual within 24 hours of administering the plasmapheresis.

21. The method of claim 1, further comprising administering intravenous immunoglobulin at a dose of 2 grams.

22. The method of claim 1, further comprising performing the method on the individual at least two times per month for at least three months, wherein two of the at least two times per month are within the same week of the month.

23. The method of claim 1, further comprising administering intravenous immunoglobulin to the individual at least two times per month.

24. The method of claim 1, wherein the exogenous particles comprise an inorganic particle or an organic particle.

25. The method of claim 24, wherein the organic particle is a polymer particle, a carbon particle, or a combination thereof.

26. The method of claim 25, wherein the polymer particle comprises plastic, polylactic-co-glycolic acid (PLGA), polyacrylonitrile, polystyrene, polyethylene, low-density polyethylene, high-density polyethylene, polypropylene, polyvinyl chloride, polyurethane, and/or polyethylene terephthalate, poly(l-aspartic acid-co-lactic acid), polyethylene glycol, poly(beta-amino ester), polybutyl cyanoacrylate, or chitosan.

27. The method of claim 26, wherein the carbon particle comprises carbon black, graphene oxide, a graphene platelet, a fullerine, a single-walled carbon nanotube, a polycyclic aromatic hydrocarbon, a lipid nanoparticle, or a multi-walled carbon nanotube.

28. The method of claim 1, wherein the exogenous particles comprise particles sized between 1 nm and 2000 nm.

29. The method of claim 1, further comprising measuring the pre-treatment level of exogenous particles, the post-treatment level of exogenous particles, or both, in a whole blood sample of the individual.

30. The method of claim 29, wherein the measuring comprises a spectroscopy method, a computerized tomography scan, an ultrasound, a positron emission tomography scan, a magnetic resonance imaging scan, flow cytometry, near-infrared spectroscopy (NIR), double shot pyrolysis gas chromatography/mass spectrometry, Fourier transform infrared (FT-IR) spectrometry, visual inspection with an optical microscope Raman spectroscopy, or surface-enhanced Raman scattering, dynamic light scattering (DLS), surface plasmon resonance, or any combination thereof.
